SEQUENCE LISTING (78 sequences)

SEQ ID NO: 1 - protein, 10 aa, Artificial Sequence
  CDR1 of BJ-005-P1/ BJ-005-P2/ BJ-005-P5/ CP-ab2
GFNITDTYMH

SEQ ID NO: 2 - protein, 10 aa, Artificial Sequence
  CDR1 of BJ-005-P3
GFNIKDNYIH

SEQ ID NO: 3 - protein, 10 aa, Artificial Sequence
  CDR1 of BJ-005-P4
GDSITSGYWN

SEQ ID NO: 4 - protein, 18 aa, Artificial Sequence
  CDR2 of BJ-005-P1/ BJ-005-P2/ BJ-005-P5/ CP-ab2
GWIDPANGHTKSDPKFQG

SEQ ID NO: 5 - protein, 18 aa, Artificial Sequence
  CDR2 of BJ-005-P3
GWIDPANTNTKYDPKFQG

SEQ ID NO: 6 - protein, 16 aa, Artificial Sequence
  CDR2 of BJ-005-P4
YISYTGSTYYNPSLKS

SEQ ID NO: 7 - protein, 13 aa, Artificial Sequence
  CDR3 of BJ-005-P1/ BJ-005-P2/ BJ-005-P5/ CP-ab2
RGDGYRYFYAMGY

SEQ ID NO: 8 - protein, 13 aa, Artificial Sequence
  CDR3 of BJ-005-P3
RGDGYRYFYAMAY

SEQ ID NO: 9 - protein, 12 aa, Artificial Sequence
  CDR3 of BJ-005-P4
GSQREWLLHTDY

SEQ ID NO: 10 - protein, 11 aa, Artificial Sequence
  BJ-005-P1/ BJ-005-P2/ BJ-005-P5/ CP-ab2CDR1′
RASQDISNYLN

SEQ ID NO: 11 - protein, 11 aa, Artificial Sequence
  CDR1′ of BJ-005-P3
RASQDISDYLN

SEQ ID NO: 12 - protein, 17 aa, Artificial Sequence
  CDR1′ of BJ-005-P4
KSSQSLLYTSNQKNSLA

SEQ ID NO: 13 - protein, 7 aa, Artificial Sequence
  CDR2′ of BJ-005-P1/ BJ-005-P2/ BJ-005-P5/ CP-ab2
YTSRLRS

SEQ ID NO: 14 - protein, 7 aa, Artificial Sequence
  CDR2′ of BJ-005-P3
HTSRLHS

SEQ ID NO: 15 - protein, 7 aa, Artificial Sequence
  CDR2′ of BJ-005-P4
WASTRES

SEQ ID NO: 16 - protein, 9 aa, Artificial Sequence
  BJ-005-P1/ BJ-005-P2/ BJ-005-P3/ BJ-005-P5/ CP-ab2CDR3′
QQGNTLPPT

SEQ ID NO: 17 - protein, 9 aa, Artificial Sequence
  CDR3′ of BJ-005-P4
QQYYGYPYT

SEQ ID NO: 18 - protein, 25 aa, Artificial Sequence
  FR1 of BJ-005-P1/BJ-005-P2/BJ-005-P3
EVQLVQSGAEVKKPGSSVKVSCAAS

SEQ ID NO: 19 - protein, 25 aa, Artificial Sequence
  FR1 of BJ-005-P4
QVQLQESGPGLVKPSETLSLTCTVT

SEQ ID NO: 20 - protein, 25 aa, Artificial Sequence [sequence omitted]

SEQ ID NO: 21 - protein, 25 aa, Artificial Sequence [sequence omitted]

SEQ ID NO: 22 - protein, 13 aa, Artificial Sequence
  FR2 of BJ-005-P1/BJ-005-P2/BJ-005-P3
WVRQAPGQGLEWI

SEQ ID NO: 23 - protein, 14 aa, Artificial Sequence
  FR2 of BJ-005-P4
WIRQPAGKGLEYMG

SEQ ID NO: 24 - protein, 13 aa, Artificial Sequence
  FR2 of BJ-005-P5
WVRQAPGKGLEWV

SEQ ID NO: 25 - protein, 13 aa, Artificial Sequence
  FR2 of CP-ab2
WVRQAPGQRLEWM

SEQ ID NO: 26 - protein, 32 aa, Artificial Sequence
  FR3 of BJ-005-P1/BJ-005-P2/BJ-005-P3
KATITADTSTNTAYMELSSLRSEDTAVYYCAR

SEQ ID NO: 27 - protein, 30 aa, Artificial Sequence
  FR3 of BJ-005-P4
RLTISRDTSKNQYSLKLSSVTAADTAVYYC

SEQ ID NO: 28 - protein, 32 aa, Artificial Sequence
  FR3 of BJ-005-P5
RFTISADTSTNTAYLQMNSLRAEDTAVYYCAR

SEQ ID NO: 29 - protein, 32 aa, Artificial Sequence
  FR3 of CP-ab2
RVTITADTSASTAYMELSSLRSEDTAVYYCAR

SEQ ID NO: 30 - protein, 11 aa, Artificial Sequence
  BJ-005-P1/BJ-005-P2/BJ-005-P3/ BJ-005-P4/ BJ-005-P5FR4
WGQGTLVTVSS

SEQ ID NO: 31 - protein, 11 aa, Artificial Sequence
  FR4 of CP-ab2
WGQGTTVTVSS

SEQ ID NO: 32 - protein, 23 aa, Artificial Sequence
  FR1′ of BJ-005-P1/BJ-005-P2/BJ-005-P3
DIQMTQSPSTLSASVGDRVTITC

SEQ ID NO: 33 - protein, 23 aa, Artificial Sequence
  FR1′ of BJ-005-P4
DIVMTQSPDSLAVSLGERATINC

SEQ ID NO: 34 - protein, 23 aa, Artificial Sequence
  FR1′ of BJ-005-P5/ CP-ab2
DIQMTQSPSSLSASVGDRVTITC

SEQ ID NO: 35 - protein, 15 aa, Artificial Sequence
  FR2 of BJ-005-P1/ CP-ab2′
WYQQKPGKAPKLLIY

SEQ ID NO: 36 - protein, 15 aa, Artificial Sequence
  FR2′ of BJ-005-P2/ BJ-005-P3
WYQQKPDQSPKLLIY

SEQ ID NO: 37 - protein, 15 aa, Artificial Sequence
  FR2′ of BJ-005-P4
WYQQKPGQPPKLLIY

SEQ ID NO: 38 - protein, 15 aa, Artificial Sequence
  FR2′ of BJ-005-P5
WYQQKPGKAPELLIY

SEQ ID NO: 39 - protein, 32 aa, Artificial Sequence
  FR3′ of BJ-005-P1/BJ-005-P2/BJ-005-P3
GVPARFSGSGSGTEYTLTISSLQPDDFATYYC

SEQ ID NO: 40 - protein, 32 aa, Artificial Sequence
  FR3′ of BJ-005-P4
GVPDRFSGSGSGTDFTLTISSLQAEDVAVYYC

SEQ ID NO: 41 - protein, 32 aa, Artificial Sequence
  FR3′ of BJ-005-P5
GVPSRFSGSGSGTDYTLTISSLQPEDFATYYC

SEQ ID NO: 42 - protein, 32 aa, Artificial Sequence
  FR3′ of CP-ab2
GVPSRFSGSGSGTDYTFTISSLQPEDIATYYC

SEQ ID NO: 43 - protein, 10 aa, Artificial Sequence
  FR4′ of BJ-005-P1/BJ-005-P2/BJ-005-P3
FGQGTKVEVK

SEQ ID NO: 44 - protein, 10 aa, Artificial Sequence
  FR4′ of BJ-005-P4/ CP-ab2
FGQGTKLEIK

SEQ ID NO: 45 - protein, 10 aa, Artificial Sequence
  FR4′ of BJ-005-P5
FGQGTKVEIK

SEQ ID NO: 46 - protein, 122 aa, Artificial Sequence
  Heavy chain variable domain of BJ-005-P1/ BJ-005-P2
EVQLVQSGAEVKKPGSSVKVSCAASGFNITDTYMHWVRQAPGQGLEWIGWIDPANGHTKSDPKFQGKATITADTSTNTAYMELSSLRSEDTAVYYCARRGDGYRYFYAMGYWGQGTLVTVSS

SEQ ID NO: 47 - protein, 122 aa, Artificial Sequence
  Heavy chain variable domain of BJ-005-P3
EVQLVQSGAEVKKPGSSVKVSCAASGFNIKDNYIHWVRQAPGQGLEWIGWIDPANTNTKYDPKFQGKATITADTSTNTAYMELSSLRSEDTAVYYCARRGDGYRYFYAMAYWGQGTLVTVSS

SEQ ID NO: 48 - protein, 118 aa, Artificial Sequence
  Heavy chain variable domain of BJ-005-P4
QVQLQESGPGLVKPSETLSLTCTVTGDSITSGYWNWIRQPAGKGLEYMGYISYTGSTYYNPSLKSRLTISRDTSKNQYSLKLSSVTAADTAVYYCGSQREWLLHTDYWGQGTLVTVSS

SEQ ID NO: 49 - protein, 122 aa, Artificial Sequence
  Heavy chain variable domain of BJ-005-P5
EVQLVESGGGLVQPGGSLRLSCAASGFNITDTYMHWVRQAPGKGLEWVGWIDPANGHTKSDPKFQGRFTISADTSTNTAYLQMNSLRAEDTAVYYCARRGDGYRYFYAMGYWGQGTLVTVSS

SEQ ID NO: 50 - protein, 122 aa, Artificial Sequence
  Heavy chain variable domain of CP-ab2
QVQLVQSGAEVKKPGASVKVSCKASGFNITDTYMHWVRQAPGQRLEWMGWIDPANGHTKSDPKFQGRVTITADTSASTAYMELSSLRSEDTAVYYCARRGDGYRYFYAMGYWGQGTTVTVSS

SEQ ID NO: 51 - protein, 107 aa, Artificial Sequence
  Light chain variable domain of BJ-005-P1
DIQMTQSPSTLSASVGDRVTITCRASQDISNYLNWYQQKPGKAPKLLIYYTSRLRSGVPARFSGSGSGTEYTLTISSLQPDDFATYYCQQGNTLPPTFGQGTKVEVK

SEQ ID NO: 52 - protein, 107 aa, Artificial Sequence
  Light chain variable domain of BJ-005-P2
DIQMTQSPSTLSASVGDRVTITCRASQDISNYLNWYQQKPDQSPKLLIYYTSRLRSGVPARFSGSGSGTEYTLTISSLQPDDFATYYCQQGNTLPPTFGQGTKVEVK

SEQ ID NO: 53 - protein, 107 aa, Artificial Sequence
  Light chain variable domain of BJ-005-P3
DIQMTQSPSTLSASVGDRVTITCRASQDISDYLNWYQQKPDQSPKLLIYHTSRLHSGVPARFSGSGSGTEYTLTISSLQPDDFATYYCQQGNTLPPTFGQGTKVEVK

SEQ ID NO: 54 - protein, 113 aa, Artificial Sequence
  Light chain variable domain of BJ-005-P4
DIVMTQSPDSLAVSLGERATINCKSSQSLLYTSNQKNSLAWYQQKPGQPPKLLIYWASTRESGVPDRFSGSGSGTDFTLTISSLQAEDVAVYYCQQYYGYPYTFGQGTKLEIK

SEQ ID NO: 55 - protein, 107 aa, Artificial Sequence
  Light chain variable domain of BJ-005-P5
DIQMTQSPSSLSASVGDRVTITCRASQDISNYLNWYQQKPGKAPELLIYYTSRLRSGVPSRFSGSGSGTDYTLTISSLQPEDFATYYCQQGNTLPPTFGQGTKVEIK

SEQ ID NO: 56 - protein, 107 aa, Artificial Sequence
  Light chain variable domain of CP-ab2
DIQMTQSPSSLSASVGDRVTITCRASQDISNYLNWYQQKPGKAPKLLIYYTSRLRSGVPSRFSGSGSGTDYTFTISSLQPEDIATYYCQQGNTLPPTFGQGTKLEIK

SEQ ID NO: 57 - protein, 329 aa, Artificial Sequence
  Heavy Chain Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPK

SEQ ID NO: 58 - protein, 107 aa, Artificial Sequence [sequence omitted]

SEQ ID NO: 59 - protein, 451 aa, Artificial Sequence
  Heavy Chain of BJ-005-P1/ BJ-005-P2
EVQLVQSGAEVKKPGSSVKVSCAASGFNITDTYMHWVRQAPGQGLEWIGWIDPANGHTKSDPKFQGKATITADTSTNTAYMELSSLRSEDTAVYYCARRGDGYRYFYAMGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPK

SEQ ID NO: 60 - protein, 451 aa, Artificial Sequence
  Heavy Chain of BJ-005-P3
EVQLVQSGAEVKKPGSSVKVSCAASGFNIKDNYIHWVRQAPGQGLEWIGWIDPANTNTKYDPKFQGKATITADTSTNTAYMELSSLRSEDTAVYYCARRGDGYRYFYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPK

SEQ ID NO: 61 - protein, 447 aa, Artificial Sequence
  Heavy Chain of BJ-005-P4
QVQLQESGPGLVKPSETLSLTCTVTGDSITSGYWNWIRQPAGKGLEYMGYISYTGSTYYNPSLKSRLTISRDTSKNQYSLKLSSVTAADTAVYYCGSQREWLLHTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPK

SEQ ID NO: 62 - protein, 451 aa, Artificial Sequence
  Heavy Chain of BJ-005-P5
EVQLVESGGGLVQPGGSLRLSCAASGFNITDTYMHWVRQAPGKGLEWVGWIDPANGHTKSDPKFQGRFTISADTSTNTAYLQMNSLRAEDTAVYYCARRGDGYRYFYAMGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPK

SEQ ID NO: 63 - protein, 451 aa, Artificial Sequence
  Heavy Chain of CP-ab2
QVQLVQSGAEVKKPGASVKVSCKASGFNITDTYMHWVRQAPGQRLEWMGWIDPANGHTKSDPKFQGRVTITADTSASTAYMELSSLRSEDTAVYYCARRGDGYRYFYAMGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPK

SEQ ID NO: 64 - protein, 214 aa, Artificial Sequence
  Light Chain of BJ-005-P1
DIQMTQSPSTLSASVGDRVTITCRASQDISNYLNWYQQKPGKAPKLLIYYTSRLRSGVPARFSGSGSGTEYTLTISSLQPDDFATYYCQQGNTLPPTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 214 aa, Artificial Sequence
  Light Chain of BJ-005-P2
DIQMTQSPSTLSASVGDRVTITCRASQDISNYLNWYQQKPDQSPKLLIYYTSRLRSGVPARFSGSGSGTEYTLTISSLQPDDFATYYCQQGNTLPPTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 214 aa, Artificial Sequence
  Light Chain of BJ-005-P3
DIQMTQSPSTLSASVGDRVTITCRASQDISDYLNWYQQKPDQSPKLLIYHTSRLHSGVPARFSGSGSGTEYTLTISSLQPDDFATYYCQQGNTLPPTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 220 aa, Artificial Sequence
  Light Chain of BJ-005-P4
DIVMTQSPDSLAVSLGERATINCKSSQSLLYTSNQKNSLAWYQQKPGQPPKLLIYWASTRESGVPDRFSGSGSGTDFTLTISSLQAEDVAVYYCQQYYGYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 214 aa, Artificial Sequence
  Light Chain of BJ-005-P5
DIQMTQSPSSLSASVGDRVTITCRASQDISNYLNWYQQKPGKAPELLIYYTSRLRSGVPSRFSGSGSGTDYTLTISSLQPEDFATYYCQQGNTLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 214 aa, Artificial Sequence
  Light Chain of CP-ab2
DIQMTQSPSSLSASVGDRVTITCRASQDISNYLNWYQQKPGKAPKLLIYYTSRLRSGVPSRFSGSGSGTDYTFTISSLQPEDIATYYCQQGNTLPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 20 aa, Artificial Sequence
  linker
GGGGSGGGGSGGGGSGGGGS

SEQ ID NO: 71 - protein, 137 aa, Artificial Sequence
  TGFRII
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 72 - protein, 602 aa, Artificial Sequence
  BJ-007Heavy chain +linker+TGFbetaRII)
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSGGGGSGGGGSGGGGTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 73 - protein, 216 aa, Artificial Sequence
  BJ-007 (Light Chain)
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 74 - protein, 608 aa, Artificial Sequence
  BJ-005-P1/ BJ-005-P2 (Heavy chain + linker +TGFbetaRII)
EVQLVQSGAEVKKPGSSVKVSCAASGFNITDTYMHWVRQAPGQGLEWIGWIDPANGHTKSDPKFQGKATITADTSTNTAYMELSSLRSEDTAVYYCARRGDGYRYFYAMGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPKGGGGSGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 75 - protein, 608 aa, Artificial Sequence
  BJ-005-P3 (Heavy chain + linker +TGFbetaRII)
EVQLVQSGAEVKKPGSSVKVSCAASGFNIKDNYIHWVRQAPGQGLEWIGWIDPANTNTKYDPKFQGKATITADTSTNTAYMELSSLRSEDTAVYYCARRGDGYRYFYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPKGGGGSGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 76 - protein, 604 aa, Artificial Sequence
  BJ-005-P4 (heavy chain + linker +TGFbetaRII)
QVQLQESGPGLVKPSETLSLTCTVTGDSITSGYWNWIRQPAGKGLEYMGYISYTGSTYYNPSLKSRLTISRDTSKNQYSLKLSSVTAADTAVYYCGSQREWLLHTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPKGGGGSGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 77 - protein, 608 aa, Artificial Sequence
  BJ-005-P5 (heavy chain + linker+TGFbetaRII)
EVQLVESGGGLVQPGGSLRLSCAASGFNITDTYMHWVRQAPGKGLEWVGWIDPANGHTKSDPKFQGRFTISADTSTNTAYLQMNSLRAEDTAVYYCARRGDGYRYFYAMGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPKGGGGSGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 78 - protein, 608 aa, Artificial Sequence
  CP-ab2 (heavy chain + linker+TGFbetaRII)
QVQLVQSGAEVKKPGASVKVSCKASGFNITDTYMHWVRQAPGQRLEWMGWIDPANGHTKSDPKFQGRVTITADTSASTAYMELSSLRSEDTAVYYCARRGDGYRYFYAMGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPKGGGGSGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

(19) United States
(12) Patent Application Publication
(10) Pub. No.: US 2024/0158504 A1
(43) Pub. Date: May 16, 2024

(54) BIFUNCTIONAL MOLECULE BINDING TO HUMAN TGFß AND PD-L1, AND USE THEREOF

(71) Applicant: BJ Bioscience Inc., Hangzhou, Zhejiang (CN)

(72) Inventors: Zhong WANG, Shanghai (CN); Haizhou ZHANG, Shanghai (CN); Ziyan ZHU, Hangzhou (CN); Yujuan LI, Suzhou (CN); Meng ZHANG, Jinan (CN); Shengdan BAI, Hangzhou (CN)

(21) Appl. No.: 18/281,632

(22) PCT Filed: Mar. 11, 2022
(86) PCT No.: PCT/CN2022/080364

§ 371 Date: Nov. 7, 2023

(30) Foreign Application Priority Data
Mar. 12, 2021 (CN) 202110272538.8

(51) Int. Cl. C07K 16/28; A61K 39/00; A61P 35/00; C07K 14/71; C12N 15/63

(52) U.S. Cl. CPC C07K 16/2827; A61P 35/00; C07K 14/71; C12N 15/63; A61K 2039/505; C07K 2317/565; C07K 2317/567

(57) ABSTRACT

Provided are a bifunctional molecule capable of binding to human transforming growth factor β (TGFβ) and human programmed death-ligand 1 (PD-L1), a pharmaceutical composition comprising the same, and use thereof treating cancer. The bifunctional molecule has high affinity for PD-L1, can significantly promote the secretion of IL-2 and IFN-γ, enhances T cell immune responses, and can block TGFβ with high specificity, and thus can be used for treating cancer. Also provided is a monoclonal antibody or an antigen-binding fragment thereof binding to human PD-L1.

### TECHNICAL FIELD

The present invention relates to a bifunctional molecule capable of binding to human transforming growth factor β (TGFβ) and human programmed death-ligand 1 (PD-L1), a pharmaceutical composition comprising the same, and use thereof in treating cancer. The present invention further relates to a monoclonal antibody or an antigen-binding fragment thereof binding to PD-L1, a pharmaceutical composition comprising the same, and use thereof in treating cancer.

### BACKGROUND

It is a research hotspot in current tumor immunotherapy to use immune checkpoints such as programmed death receptor 1 (PD-1)/programmed death receptor ligand 1 (PD-L1) as targets in a immunotherapy. Since the objective response rate (ORR) of a PD-1/PD-L1 monoclonal antibody used alone is not high, or it is required to screen out a PD-L1-expressing population, now the PD-1/PD-L1 monoclonal antibody basically needs to be used in combination with a chemotherapy or radiotherapy method, and such a combination may bring about increase in toxicity and reduction in patient compliance, and some patients still have no response to treatment.

M7824 (MSB0011359C) is an innovative bifunctional fusion protein developed by Merck, which targets PD-L1/TGF-β respectively. Preclinical studies have confirmed that there may be complementary interaction between PD-L1 and TGFβ pathways, and it also shows that M7824 has the ability to reverse the mesenchymal transition of cancer cells and enhance the response to chemotherapy, and its dual inhibition can enhance the anti-tumor activity. Importantly, targeting PD-L1 and TGFβ simultaneously in a tumor microenvironment is also effective for immune excluded cold tumors. In preclinical studies, M7824 has stronger anti-tumor activity compared with an anti-PD-L1 monoclonal antibody used alone or an anti-PD-L1 monoclonal antibody used simultaneously with an anti-TGF-β monoclonal antibody.

Although M7824 has made remarkable progress in the research of tumor therapy, it is still necessary in clinical to provide more bifunctional molecules capable of binding to human TGFβ and PD-L1 to meet the market demand.

### SUMMARY

The present invention provides a bifunctional molecule binding to human TGFβ and PD-L1, wherein the bifunctional molecule comprises: (a) human TGFβRII or a functional fragment thereof capable of binding to human TGFβ, and (b) an antibody or an antigen-binding fragment thereof binding to human PD-L1, wherein the antibody or the antigen-binding fragment thereof comprises: (i) heavy chain complementarity determining regions CDR1, CDR2 and CDR3, wherein the CDR1 comprises an amino acid sequence selected from SEQ ID NO: 1, SEQ ID NO: 2 and SEQ ID NO: 3 or an equivalent variant thereof, the CDR2 comprises an amino acid sequence selected from SEQ ID NO: 4, SEQ ID NO: 5 and SEQ ID NO: 6 or an equivalent variant thereof, and the CDR3 comprises an amino acid sequence selected from SEQ ID NO: 7, SEQ ID NO: 8 and SEQ ID NO: 9 or an equivalent variant thereof; and (ii) light chain complementarity determining regions CDR1′, CDR2′ and CDR3′, wherein the CDR1′ comprises an amino acid sequence selected from SEQ ID NO: 10, SEQ ID NO: 11 and SEQ ID NO: 12 or an equivalent variant thereof, the CDR2′ comprises an amino acid sequence selected from SEQ ID NO: 13, SEQ ID NO: 14 and SEQ ID NO: 15 or an equivalent variant thereof, and the CDR3′ comprises an amino acid as shown in SEQ ID NO: 16 or SEQ ID NO: 17 or an equivalent variant thereof. In some embodiments, the antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof. In some embodiments, a C-terminal of the antibody or the antigen-binding fragment thereof is connected with an N-terminal of the human TGFβRII or a functional fragment thereof capable of binding to human TGFβ through a linker. In some embodiments, an amino acid sequence of the linker is as shown in SEQ ID NO: 70. In some embodiments, an amino acid sequence of the functional fragment of the human TGFβRII capable of binding to human TGFβ is as shown in SEQ ID NO: 71. In some embodiments, the bifunctional molecule has a three-dimensional spatial structure.

In another aspect, the present invention provides a bifunctional molecule binding to human TGFβ and PD-L1, wherein the bifunctional molecule comprises: (a) human TGFβRII or a functional fragment thereof capable of binding to human TGFβ, and (b) an antibody or an antigen-binding fragment thereof binding to human PD-L1, wherein the antibody or the antigen-binding fragment thereof comprises: (i) heavy chain complementarity determining regions CDR1, CDR2 and CDR3, wherein the CDR1 comprises an amino acid sequence selected from SEQ ID NO: 1, SEQ ID NO: 2 and SEQ ID NO: 3 or an equivalent variant thereof, the CDR2 comprises an amino acid sequence selected from SEQ ID NO: 4, SEQ ID NO: 5 and SEQ ID NO: 6 or an equivalent variant thereof, and the CDR3 comprises an amino acid sequence selected from SEQ ID NO: 7, SEQ ID NO: 8 and SEQ ID NO: 9 or an equivalent variant thereof; (ii) heavy chain framework regions FR1, FR2, FR3 and FR4, wherein the FR1 comprises an amino acid sequence selected from SEQ ID NO: 18, SEQ ID NO: 19, SEQ ID NO: 20 and SEQ ID NO: 21 or an equivalent variant thereof, the FR2 comprises an amino acid sequence selected from SEQ ID NO: 22, SEQ ID NO: 23, SEQ ID NO: 24 and SEQ ID NO: 25 or an equivalent variant thereof, the FR3 comprises an amino acid sequence selected from SEQ ID NO: 26, SEQ ID NO: 27, SEQ ID NO: 28 and SEQ ID NO: 29 or an equivalent variant thereof, and the FR4 comprises an amino acid sequence as shown in SEQ ID NO: 30 or SEQ ID NO: 31 or an equivalent variant thereof; (iii) light chain complementarity determining regions CDR1′, CDR2′ and CDR3′, wherein the CDR1′ comprises an amino acid sequence selected from SEQ ID NO: 10, SEQ ID NO: 11 and SEQ ID NO: 12 or an equivalent variant thereof, the CDR2′ comprises an amino acid sequence selected from SEQ ID NO: 13, SEQ ID NO: 14 and SEQ ID NO: 15 or an equivalent variant thereof, and the CDR3′ comprises an amino acid sequence as shown in SEQ ID NO: 16 or SEQ ID NO: 17 or an equivalent variant thereof; and (iv) light chain framework regions FR1′, FR2′, FR3′ and FR4′, wherein the FR1′ comprises an amino acid sequence selected from SEQ ID NO: 32, SEQ ID NO: 33 and SEQ ID NO: 34 or an equivalent variant thereof, the FR2′ comprises an amino acid sequence selected from SEQ ID NO: 35, SEQ ID NO: 36, SEQ ID NO: 37 and SEQ ID NO: 38 or an equivalent variant thereof, the FR3′ comprises an amino acid sequence selected from SEQ ID NO: 39, SEQ ID NO: 40, SEQ ID NO: 41 and SEQ ID NO: 42 or an equivalent variant thereof, and the FR4′ comprises an amino acid sequence selected from SEQ ID NO: 43, SEQ ID NO: 44 and SEQ ID NO: 45 or an equivalent variant thereof. In some embodiments, the antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof. In some embodiments, a C-terminal of the antibody or the antigen-binding fragment thereof is connected with an N-terminal of the human TGFβRII or a functional fragment thereof capable of binding to human TGFβ through a linker. In some embodiments, an amino acid sequence of the linker is as shown in SEQ ID NO: 70. In some embodiments, an amino acid sequence of the human TGFβRII is as shown in SEQ ID NO: 71. In some embodiments, the bifunctional molecule has a three-dimensional spatial structure.

In another aspect, the present invention provides a bifunctional molecule binding to human TGFβ and PD-L1, wherein the bifunctional molecule comprises: (a) human TGFβRII or a functional fragment thereof capable of binding to human TGFβ, and (b) an antibody or an antigen-binding fragment thereof binding to human PD-L1, wherein the antibody or the antigen-binding fragment thereof comprises a heavy chain variable region (VH) and a light chain variable region (VL), wherein the heavy chain variable region comprises an amino acid sequence selected from SEQ ID NO: 46, SEQ ID NO: 47, SEQ ID NO: 48, SEQ ID NO: 49 and SEQ ID NO: 50 or an equivalent variant thereof, and the light chain variable region comprises an amino acid sequence selected from SEQ ID NO: 51, SEQ ID NO: 52, SEQ ID NO: 53, SEQ ID NO: 54, SEQ ID NO: 55 and SEQ ID NO: 56 or an equivalent variant thereof. In some embodiments, the antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof. In some embodiments, a C-terminal of the antibody or the antigen-binding fragment thereof is connected with an N-terminal of the human TGFβRII or a functional fragment thereof capable of binding to human TGFβ through a linker. In some embodiments, an amino acid sequence of the linker is as shown in SEQ ID NO: 70. In some embodiments, an amino acid sequence of the functional fragment of the human TGFβRII capable of binding to human TGFβ is as shown in SEQ ID NO: 71. In some embodiments, the bifunctional molecule has a three-dimensional spatial structure.

In another aspect, the present invention provides a bifunctional molecule binding to human TGFβ and PD-L1, wherein the bifunctional molecule comprises: (a) human TGFβRII or a functional fragment thereof capable of binding to human TGFβ, and (b) an antibody or an antigen-binding fragment thereof binding to human PD-L1, wherein the antibody or the antigen-binding fragment thereof comprises a heavy chain and a light chain, wherein the heavy chain comprises an amino acid sequence selected from SEQ ID NO: 59, SEQ ID NO: 60, SEQ ID NO: 61, SEQ ID NO: 62 and SEQ ID NO: 63 or an equivalent variant thereof, and the light chain comprises an amino acid sequence selected from SEQ ID NO: 64, SEQ ID NO: 65, SEQ ID NO: 66, SEQ ID NO: 67, SEQ ID NO: 68 and SEQ ID NO: 69 or an equivalent variant thereof. In some embodiments, a C-terminal of the antibody or the antigen-binding fragment thereof is connected with an N-terminal of the human TGFβRII or a functional fragment thereof capable of binding to human TGFβ through a linker. In some embodiments, an amino acid sequence of the linker is as shown in SEQ ID NO: 70. In some embodiments, an amino acid sequence of the functional fragment of the human TGFβRII is as shown in SEQ ID NO: 71. In some embodiments, the bifunctional molecule has a three-dimensional spatial structure.

In another aspect, the present invention further provides a pharmaceutical composition for treating a cancer in a subject, which comprises the bifunctional molecule binding to human TGFβ and PD-L1 of the present invention, and a pharmaceutically acceptable carrier.

In another aspect, the present invention further provides a pharmaceutical composition for treating a cancer in a subject, which comprises the bifunctional molecule binding to human TGFβ and PD-L1 of the present invention, and a second therapeutic agent.

In another aspect, the present invention further provides use of the bifunctional molecule binding to human TGFβ and PD-L1 of the present invention in the manufacture of a medicament for treating a cancer.

In another aspect, the present invention further provides a nucleotide sequence encoding the aforementioned bifunctional molecule.

In another aspect, the present invention further provides a vector including the aforementioned nucleotide sequence.

In another aspect, the present invention further provides a non-human host cell including the aforementioned vector. Additionally, the present invention further provides a cell line for producing the bifunctional molecule of the present invention, a recombinant expression vector comprising the nucleotide of the present invention, and a method for preparing the antibody by culturing an antibody-producing cell line.

In another aspect, the present invention further provides a method for treating a cancer in a subject, which comprises administering to the subject a therapeutically effective amount of any one of the bifunctional molecules of the present invention or any one of the pharmaceutical compositions of the present invention.

The bifunctional molecule provided by the present invention binds to human PD-L1 with high affinity, significantly promotes the secretion of IL-2 and IFN-γ, enhances T cell immune responses, and blocks TGFβ with high specificity. Experiments prove that these bifunctional molecules have prolonged existence time in blood and significantly inhibit the growth of tumors.

In another aspect, the present invention further provides an monoclonal antibody or an antigen-binding fragment thereof binding to human PD-L1, wherein the monoclonal antibody or the antigen-binding fragment thereof comprises: (i) heavy chain complementarity determining regions CDR1, CDR2 and CDR3, wherein the CDR1 comprises an amino acid sequence as shown in SEQ ID NO: 2 or SEQ ID NO: 3 or an equivalent variant thereof, the CDR2 comprises an amino acid sequence as shown in SEQ ID NO: 5 or SEQ ID NO: 6 or an equivalent variant thereof; and the CDR3 comprises an amino acid sequence as shown in SEQ ID NO: 8 or SEQ ID NO: 9 or an equivalent variant thereof; and (ii) light chain complementarity determining regions CDR1′, CDR2′ and CDR3′, wherein the CDR1′ comprises an amino acid sequence as shown in SEQ ID NO: 11 or SEQ ID NO: 12 or an equivalent variant thereof, the CDR2′ comprises an amino acid sequence as shown in SEQ ID NO: 14 or SEQ ID NO: 15 or an equivalent variant thereof, and the CDR3′ comprises an amino acid sequence as shown in SEQ ID NO: 16 or SEQ ID NO: 17 or an equivalent variant thereof. In some embodiments, the monoclonal antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof.

In another aspect, the present invention further provides an monoclonal antibody or an antigen-binding fragment thereof binding to human PD-L1, wherein the monoclonal antibody or the antigen-binding fragment thereof comprises: (i) heavy chain complementarity determining regions CDR1, CDR2 and CDR3, wherein the CDR1 comprises an amino acid sequence as shown in SEQ ID NO: 2 or SEQ ID NO: 3 or an equivalent variant thereof, the CDR2 comprises an amino acid sequence as shown in SEQ ID NO: 5 or SEQ ID NO: 6 or an equivalent variant thereof, and the CDR3 comprises an amino acid sequence as shown in SEQ ID NO: 8 or SEQ ID NO: 9 or an equivalent variant thereof; (ii) heavy chain framework regions FR1, FR2, FR3 and FR4, wherein the FR1 comprises an amino acid sequence as shown in SEQ ID NO: 18 or SEQ ID NO: 19 or an equivalent variant thereof, the FR2 comprises an amino acid sequence as shown in SEQ ID NO: 22 or SEQ ID NO: 23 or an equivalent variant thereof, the FR3 comprises an amino acid sequence as shown in SEQ ID NO: 26 or SEQ ID NO: 27 or an equivalent variant thereof, and the FR4 comprises an amino acid sequence as shown in SEQ ID NO: 30 or an equivalent variant thereof; (iii) light chain complementarity determining regions CDR1′, CDR2′ and CDR3′, wherein the CDR1′ comprises an amino acid sequence as shown in SEQ ID NO: 11 or SEQ ID NO: 12 or an equivalent variant thereof, the CDR2′ comprises an amino acid sequence as shown in SEQ ID NO: 14 or SEQ ID NO: 15 or an equivalent variant thereof, and the CDR3′ comprises an amino acid sequence as shown in SEQ ID NO: 16 or SEQ ID NO: 17 or an equivalent variant thereof; and (iv) light chain framework regions FR1′, FR2′, FR3′ and FR4′, wherein the FR1′ comprises an amino acid sequence as shown in SEQ ID NO: 32 or SEQ ID NO: 33 or an equivalent variant thereof, the FR2′ comprises an amino acid sequence as shown in SEQ ID NO: 36 or SEQ ID NO: 37 or an equivalent variant thereof, the FR3′ comprises an amino acid sequence as shown in SEQ ID NO: 39 or SEQ ID NO: 40 or an equivalent variant thereof, and the FR4′ comprises an amino acid sequence as shown in SEQ ID NO: 43 or SEQ ID NO: 44 or an equivalent variant thereof. In some embodiments, the monoclonal antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof.

In another aspect, the present invention provides a monoclonal antibody or an antigen-binding fragment thereof binding to human PD-L1, wherein the monoclonal antibody or the antigen-binding fragment thereof comprises a heavy chain variable region (VH) and a light chain variable region (VL), wherein the heavy chain variable region comprises an amino acid sequence as shown in SEQ ID NO: 47 or SEQ ID NO: 48 or an equivalent variant thereof, and the light chain variable region comprises an amino acid sequence as shown in SEQ ID NO: 53 or SEQ ID NO: 54 or an equivalent variant thereof. In some embodiments, the monoclonal antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof.

In another aspect, the present invention provides a monoclonal antibody or an antigen-binding fragment thereof binding to human PD-L1, wherein the monoclonal antibody or the antigen-binding fragment thereof comprises a heavy chain and a light chain, wherein the heavy chain comprises an amino acid sequence as shown in SEQ ID NO: 60 or SEQ ID NO: 61 or an equivalent variant thereof, and the light chain comprises an amino acid sequence as shown in SEQ ID NO: 66 or SEQ ID NO: 67 or an equivalent variant thereof.

In another aspect, the present invention further provides a pharmaceutical composition for treating a cancer in a subject, which comprises a monoclonal antibody or an antigen-binding fragment thereof binding to human PD-L1 of the present invention, and a pharmaceutically acceptable carrier.

In another aspect, the present invention further provides a pharmaceutical composition for treating a cancer in a subject, which comprises a monoclonal antibody or an antigen-binding fragment thereof binding to human PD-L1 of the present invention, and a second therapeutic agent.

In another aspect, the present invention further provides use of the monoclonal antibody or an antigen-binding fragment thereof binding to human PD-L1 of the present invention in the manufacture of a medicament for treating a cancer.

In another aspect, the present invention further provides a nucleotide sequence encoding the aforementioned monoclonal antibody or an antigen-binding fragment thereof.

In another aspect, the present invention further provides a vector including the aforementioned nucleotide sequence.

In another aspect, the present invention further provides a non-human host cell including the aforementioned vector. Additionally, the present invention further provides a cell line for producing the monoclonal antibody or an antigen-binding fragment thereof of the present invention, a recombinant expression vector including the nucleotide of the present invention, and a method for preparing the antibody by culturing an antibody-producing cell line.

In another aspect, the present invention further provides a method for treating a cancer in a subject, which comprises administering to the subject a therapeutically effective amount of any one of the monoclonal antibodies or antigen-binding fragments thereof of the present invention or any one of the pharmaceutical compositions of the present invention.

The monoclonal antibody provided by the present invention binds to human PD-L1 with high affinity, significantly promotes the secretion of IL-2 and IFN-γ, and enhances T cell immune responses. These antibodies can be used as antineoplastic agents, immunopotentiating agents, or as diagnostic reagents for detecting human PD-L1 in blood or tissues of patients with cancer or other diseases, or as a part of a bifunctional molecule (e.g., the bifunctional molecule targeting PD-L1 and human TGFβ as described in the present invention) to bind to human PD-L1.

### BRIEF DESCRIPTION OF DRAWINGS

FIG. 1 is a gel electrophoresis diagram of a purified bifunctional molecule.

FIG. 2 shows that CP-ab1 (BJ-007), CP-ab2 (14H2/14L2), CP-ab3 (BJ-005-P2) and CP-ab4 (BJ-005-P5) all promote the release of IL-2.

FIG. 3 shows that CP-ab1 (BJ-007), CP-ab2 (14H2/14L2), CP-ab3 (BJ-005-P2) and CP-ab4 (BJ-005-P5) all promote the release of IFN-γ.

FIG. 4 shows that the specificity of BJ-005-P1, BJ-005-P2, BJ-005-P3 and BJ-005-P4 is significantly stronger than that of the control.

FIG. 5 shows a change in concentration of 1 mg/kg of BJ-005-P5 in a rat.

FIG. 6 shows a change in concentration of 5 mg/kg of BJ-005-P5 in a rat.

FIG. 7 shows a change in concentration of 1 mg/kg of BJ-007 in a rat.

FIG. 8 shows a change in concentration of 5 mg/kg of BJ-007 in a rat.

FIG. 9 shows an effect of BJ-005-P5 on tumor growth.

### DETAILED DESCRIPTION

### PD-L1

Programmed death-ligand 1 (PD-L1), also known as cluster of differentiation 274 (CD274) or B7 homolog 1 (B7-H1), belongs to the tumor necrosis factor superfamily. It is a type I transmembrane glycoprotein consisting of 290 amino acid residues, including an IgV-like region, an IgC-like region, a transmembrane hydrophobic region and an intracellular tail of 30 amino acids, with a complete molecular weight of 40 kDa. Like other B7 family members, PD-L1 can also provide a costimulatory signal to T cells. PD-L1 mRNA is expressed in almost all tissues, but PD-L1 protein is continuously expressed in only a few tissues, including liver, lung, tonsils and immune amnesty organizations such as eyes, placenta and the like. PD-L1 is also expressed in activated T cells, B cells, monocytes, dendritic cells and macrophages, etc. Under normal physiological conditions, PD-L1 mRNA is under strict post-transcriptional regulation, but PD-L1 protein is abundantly expressed on the cell surfaces of various types of human cancers.

A receptor of PD-L1 is PD-1. Programmed death-1 (PD-1) is a member of CD28 superfamily, which also includes CD28, CTLA-4, ICOS and BTLA. PD-1 is a type I transmembrane protein of immunoglobulin superfamily, has 288 amino acids, and is one of the main known immune checkpoints. It is mainly expressed on the surfaces of immune cells such as CD4+ T cells, CD8+ T cells, NKT cells, B cells and activated monocytes. The binding of PD-L1 and PD-1 can limit the interaction between antigen presenting cells or dendritic cells and T cells, further inhibit the metabolism of T cells, inhibit the secretion of Bcl-X2 of anti-apoptotic cells, reduce the secretion of effector cytokines IL-2 and IFN-γ, and induce depletion and apoptosis of T cells, thereby reducing the immune response in which immune T cells participate and exercising a negative regulatory function.

The expression of PD-L1 at a tumor site can protect tumor cells from injury in many ways. Studies have shown that the tumor cell-associated PD-L1 can increase the apoptosis of tumor-specific T cells, while a PD-L1 monoclonal antibody can weaken this effect. In cancer, PD-L1 and PD-1/PD-L1 pathways can protect tumors from cytotoxic T cells, finally inhibit the anti-tumor immune response by inactivating the cytotoxic T cells in a tumor microenvironment, and prevent the stimulation and activation of new T cells in lymph nodes and subsequent recruitment to the tumors. PD-L1 not only is a ligand of PD-1, but also can be used as a receptor to transmit reverse signals to protect tumor cells from apoptosis induced by FAS-FASL and the like anti-tumor pathways.

PD-L1 plays an important role in tumor immunity by increasing the apoptosis of antigen-specific T cell clones. It has been found that PD-L1 is expressed in the tissues of patients with various tumors, including non-small cell lung cancer, lung cancer, gastric cancer, colon cancer, liver cancer, intrahepatic cholangiocarcinoma, pancreatic cancer, ovarian cancer, breast cancer, cervical cancer, head and neck squamous cell carcinoma, nasopharyngeal cancer, esophageal cancer, bladder cancer, skin cancer, renal cell carcinoma, oral squamous cell carcinoma, urethral epithelial cell carcinoma and the like. In the process of cell malignant transformation, new protein molecules will be produced due to gene mutation and the like. Some peptide segments of these proteins after degraded in cells can be expressed on the cell surfaces to become tumor antigens. The immune system can identify tumor antigens and clear up tumor cells through immune monitoring, and the tumor cells can utilize PD-L1 to escape immune attacks.

### TGFβ

Transforming growth factor-β (TGF-β) is a cytokine that effectively suppress immunity, which has effects on most immune cells (dendritic cells, macrophages, natural killer cells, CD4+ and CD8+ cells). TGFβ also stimulates the differentiation of immunosuppressive regulatory T cells. There are three subtypes of TGFβ, namely TGFβ1, TGFβ2 and TGFβ3 respectively, which all can bind to type 2 TGFβ receptor (TGFβRII).

TGFβ plays a very important role in both the occurrence and development of tumors, and TGFβ is a tumor promoting factor in the later stage of tumors. In the late stage of tumors, most tumor cells can secrete TGFβ. Once the level of TGFβ rises, it will block the differentiation of immature T cells into Th1 cells, promote their transformation into Treg subsets, and inhibit the antigen presenting function of dendritic cells, thereby leading to the immune escape of the tumor cells. With the development of the tumors, the TGFβ receptor or a downstream Smad gene mutation thereof accumulates in tumor cells, and its inhibitory effect is weakened. Also, a TGFβ signal promotes epithelial mesenchymal transition, which process transforms polar epithelial cells into interstitial cells with mobility, thereby gaining the ability of invasion and migration, and tumor migration is one of the important causes of the death of cancer patients.

Both TGFβ ligand and receptor are intensively studied as therapeutic targets. There are three ligand isoforms, TGFβ1, TGFβ2 and TGFβ3, which all exist in the form of homodimers. There are also three TGFβ receptors (TGFβR), which are called TGFβRI, TGFβRII and TGFβRIII. TGFβRI is a signal transduction chain which cannot bind to the ligand. TGFβRII binds to the ligands TGFβ1 and TGFβ3 with high affinity, but does not bind to TGFβ2. The TGFβRII/TGFβ complex recruits TGFβRI to form a signal transduction complex. TGFβRIII is a positive regulator for TGFβ binding to a signal transduction receptor thereof, and binds all of the three TGFβ isoforms with high affinity. On the cell surface, the TGFβ/TGFβRIII complex binds to TGFβRII, and then recruits TGFβRI, which replaces the TGFβRIII to form a signal transduction complex.

Although these three different TGFβ isoforms are all transduced by the same receptor signal, they are known to have different expression patterns and their in vivo functions do not overlap. Studies have shown that TGFβ1 and TGFβ2 play major roles in tumor microenvironment and cardiac physiology, respectively. Therapeutic agents that neutralize TGFβ1 rather than TGFβ2 can minimize cardiotoxicity without affecting anti-tumor activity, thereby providing an optimal therapeutic index. Therefore, an anti-PD-L1/TGFβ fusion protein lacks toxicity, including cardiotoxicity, as confirmed by the prior art.

A therapeutic method for neutralizing TGFβ includes employing an extracellular domain of a TGFβ receptor and a neutralizing antibody. Soluble TGFβRIII seems to be an obvious choice because it binds to all of the three TGFβ ligands. However, TGFβRIII, which is naturally produced in the form of 280-330 kD glycosaminoglycan (GAG)-glycoprotein, has an extracellular domain of 762 amino acid residues, which is a very complicated protein for the development of biotherapeutics. A GAG-deficient soluble TGFβRIII can be produced in insect cells and is shown to be a powerful TGFβ neutralizing agent. Two separate binding domains of TGFβRIII (related to endothelial glycoprotein and related to uromodulin) can be expressed independently, but their affinity is shown to be 20-100 times lower than that of the soluble TGFβRIII, and they have little neutralizing activity. On the other hand, the extracellular domain of TGFβRII is only 136 amino acid residues long, and it can be produced in the form of a 25-35 kD glycosylated protein. Studies have shown that a recombinant soluble TGFβRII binds to TGFβ1 with a KD of 200 μM, which is basically similar to the KD of 50 μM for the full-length TGFβRII on a cell.

Another way to neutralize all of the three TGFβ ligand isoforms is to screen a pan-neutralizing anti-TGFβ antibody, or an anti-receptor antibody that blocks the receptor from binding to TGFβ1, TGF132 and TGF133. However, up to now, most of the research on TGFβ-targeted anticancer therapy, including generally highly toxic small molecular inhibitors for TGFβ signal transduction, is in the preclinical stage, and the obtained anti-tumor effect is limited.

The bifunctional molecule binding to human TGF-β and PD-L1 of the present invention, comprises a human TGFβ receptor II (TGFβRII) or a functional fragment thereof capable of binding to TGFβ. In an embodiment, the functional fragment is a soluble part of human TGFβ receptor type 2 isotype A, which can bind to TGFβ. In another embodiment, the functional fragment comprises amino acids 73-184 of human TGFβ receptor type 2 isotype A. In another embodiment, the functional fragment comprises amino acids 24-184 of human TGFβ receptor type 2 isotype A. In another embodiment, the functional fragment is a soluble part of human TGFβ receptor type 2 isotype B, which can bind to TGFβ. In another embodiment, the functional fragment comprises amino acids 48-159 of human TGFβ receptor type 2 isotype B. In another embodiment, the functional fragment comprises amino acids 23-159 of human TGFβ receptor type 2 isotype B (SEQ ID NO. 71). In another embodiment, the functional fragment comprises amino acids 24-159 of human TGFβ receptor type 2 isotype B. In another embodiment, the functional fragment comprises amino acids 24-105 of human TGFβ receptor type 2 isotype B.

### Antibody Fragment

As used herein, the term “antibody” refers to any form of antibody that exhibits a desired biological activity (for example inhibiting the binding of a ligand to a receptor thereof, or inhibiting receptor signal transduction induced by a ligand). An “antibody fragment” and an “antigen-binding fragment” refer to an antigen-binding fragment of an antibody and an antibody analogue, which generally include at least a portion of the antigen-binding region or variable region (e.g. one or more CDRs) of a parental antibody. The antibody fragment retains at least some of the binding specificity of the parental antibody. Typically, the antibody fragment retains at least 10% of the binding activity of the parental antibody when the activity is expressed on a molar basis. Preferably, the antibody fragment retains at least 20%, 50%, 70%, 80%, 90%, 95% or 100% or more of the binding affinity of the parent antibody for a target. Thus, as used herein, examples of the antibody fragment include, but are not limited to: Fab, Fab′, F(ab′)₂ and a Fv fragment; a double antibody; a linear antibody; a single chain antibody molecule, e.g. sc-Fv; a nano antibody; a domain antibody; and a multispecific antibody formed by antibody fragments.

A “Fab fragment” consists of one light chain and the CH1 and variable region of one heavy chain. The heavy chain of the Fab molecule cannot form a disulfide bond with another heavy chain molecule. A “Fc” region contains two heavy chain fragments including the CH1 and CH2 domains of an antibody. The two heavy chain fragments are held together by two or more disulfide bonds and by the hydrophobic interaction of the CH3 domain.

A “Fab′ fragment” contains one light chain, and part of one heavy chain including VH and CH1 domains and the region between the CH1 and CH2 domains, so that an interchain disulfide bond can be formed between the two heavy chains of two Fab′ fragments to form an F(ab′)2 molecule.

A “F(ab′)₂ fragment” contains two light chains and two heavy chains including part of the the constant region between the CH1 and CH2 domains, thereby forming an interchain disulfide bond between the two heavy chains. Therefore, the F(ab′)2 fragment consists of two Fab′ fragments held together by the disulfide bond between the two heavy chains. A “Fv region” includes variable regions from both heavy and light chains, but lacks a constant region.

A “single-chain Fv antibody” (or “scFv antibody”) refers to an antibody fragment including VH and VL domains of an antibody, wherein these domains exist in a single polypeptide chain. In general, a Fv polypeptide includes an additional polypeptide linker between the VH and VL domains, which enables scFv to form a structure required for antigen binding.

As used herein, the term “humanized antibody” refers to an antibody that includes CDRs of an antibody derived from a mammal other than human, as well as framework regions (FR) and constant regions of a human antibody.

### Anti-PD-L1 Antibody

The antibody or an antigen-binding fragment thereof of the present invention can be any anti-PD-L1 antibody or an antigen-binding fragment thereof known in the art.

In an aspect of the present invention, the antibody or an antigen-binding fragment thereof comprises: (i) heavy chain complementarity determining regions CDR1, CDR2 and CDR3, wherein the CDR1 comprises an amino acid sequence selected from SEQ ID NO: 1, SEQ ID NO: 2 and SEQ ID NO: 3 or an equivalent variant thereof, the CDR2 comprises an amino acid sequence selected from SEQ ID NO: 4, SEQ ID NO: 5 and SEQ ID NO: 6 or an equivalent variant thereof, and the CDR3 comprises an amino acid sequence selected from SEQ ID NO: 7, SEQ ID NO: 8 and SEQ ID NO: 9 or an equivalent variant thereof; and (ii) light chain complementarity determining regions CDR1′, CDR2′ and CDR3′, wherein the CDR1′ comprises an amino acid sequence selected from SEQ ID NO: 10, SEQ ID NO: 11 and SEQ ID NO: 12 or an equivalent variant thereof, the CDR2′ comprises an amino acid sequence selected from SEQ ID NO: 13, SEQ ID NO: 14 and SEQ ID NO: 15 or an equivalent variant thereof, and the CDR3′ comprises an amino acid sequence as shown in SEQ ID NO: 16 or SEQ ID NO: 17 or an equivalent variant thereof. In some embodiments, the antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof. In some embodiments, the antigen binding fragment is selected from scFv, (scFv)₂, Fab, Fab′ or F(ab′)₂ of a PD-L1 antibody.

In some embodiments, for the anti-PD-L1 antibody part of the bifunctional molecule, the CDR1 comprises an amino acid sequence as shown in SEQ ID NO: 1 or an equivalent variant thereof, the CDR2 comprises an amino acid sequence as shown in SEQ ID NO: 4 or an equivalent variant thereof, the CDR3 comprises an amino acid sequence as shown in SEQ ID NO: 7 or an equivalent variant thereof, the CDR1′ comprises an amino acid sequence as shown in SEQ ID NO: 10 or an equivalent variant thereof, the CDR2′ comprises an amino acid sequence as shown in SEQ ID NO: 13 or an equivalent variant thereof, and the CDR3′ comprises an amino acid sequence as shown in SEQ ID NO: 16 or an equivalent variant thereof.

In some embodiments, for the anti-PD-L1 antibody part of the bifunctional molecule, the CDR1 comprises an amino acid sequence as shown in SEQ ID NO: 2 or an equivalent variant thereof, the CDR2 comprises an amino acid sequence as shown in SEQ ID NO: 5 or an equivalent variant thereof, the CDR3 comprises an amino acid sequence as shown in SEQ ID NO: 8 or an equivalent variant thereof, the CDR1′ comprises an amino acid sequence as shown in SEQ ID NO: 11 or an equivalent variant thereof, the CDR2′ comprises an amino acid sequence as shown in SEQ ID NO: 14 or an equivalent variant thereof, and the CDR3′ comprises an amino acid sequence as shown in SEQ ID NO: 16 or an equivalent variant thereof.

In some embodiments, for the anti-PD-L1 antibody part of the bifunctional molecule, the CDR1 comprises an amino acid sequence as shown in SEQ ID NO: 3 or an equivalent variant thereof, the CDR2 comprises an amino acid sequence as shown in SEQ ID NO: 6 or an equivalent variant thereof, the CDR3 comprises an amino acid sequence as shown in SEQ ID NO: 9 or an equivalent variant thereof, the CDR1′ comprises an amino acid sequence as shown in SEQ ID NO: 12 or an equivalent variant thereof, the CDR2′ comprises an amino acid sequence as shown in SEQ ID NO: 15 or an equivalent variant thereof, and the CDR3′ comprises an amino acid sequence as shown in SEQ ID NO: 19 or an equivalent variant thereof.

In another aspect of the present invention, the anti-PD-L1 antibody or an antigen-binding fragment thereof of the bifunctional molecule comprises: (i) heavy chain complementarity determining regions CDR1, CDR2 and CDR3, wherein the CDR1 comprises an amino acid sequence selected from SEQ ID NO: 1, SEQ ID NO: 2 and SEQ ID NO: 3 or an equivalent variant thereof, the CDR2 comprises an amino acid sequence selected from SEQ ID NO: 4, SEQ ID NO: 5 and SEQ ID NO: 6 or an equivalent variant thereof, and the CDR3 comprises an amino acid sequence selected from SEQ ID NO: 7, SEQ ID NO: 8 and SEQ ID NO: 9 or an equivalent variant thereof; (ii) heavy chain framework regions FR1, FR2, FR3 and FR4, wherein the FR1 comprises an amino acid sequence selected from SEQ ID NO: 18, SEQ ID NO: 19, SEQ ID NO: 20 and SEQ ID NO: 21 or an equivalent variant thereof, the FR2 comprises an amino acid sequence selected from SEQ ID NO: 22, SEQ ID NO: 23, SEQ ID NO: 24 and SEQ ID NO: 25 or an equivalent variant thereof, the FR3 comprises an amino acid sequence selected from SEQ ID NO: 26, SEQ ID NO: 27, SEQ ID NO: 28 and SEQ ID NO: 29 or an equivalent variant thereof, and the FR4 comprises an amino acid sequence as shown in SEQ ID NO: 30 or SEQ ID NO: 31 or an equivalent variant thereof; (iii) light chain complementarity determining regions CDR1′, CDR2′ and CDR3′, wherein the CDR1′ comprises an amino acid sequence selected from SEQ ID NO: 10, SEQ ID NO: 11 and SEQ ID NO: 12 or an equivalent variant thereof, the CDR2′ comprises an amino acid sequence selected from SEQ ID NO: 13, SEQ ID NO: 14 and SEQ ID NO: 15 or an equivalent variant thereof, and the CDR3′ comprises an amino acid sequence as shown in SEQ ID NO: 16 or SEQ ID NO: 17 or an equivalent variant thereof; and (iv) light chain framework regions FR1′, FR2′, FR3′ and FR4′, wherein the FR1′ comprises an amino acid sequence selected from SEQ ID NO: 32, SEQ ID NO: 33 and SEQ ID NO: 34 or an equivalent variant thereof, the FR2′ comprises an amino acid sequence selected from SEQ ID NO: 35, SEQ ID NO: 36, SEQ ID NO: 37 and SEQ ID NO: 38 or an equivalent variant thereof, the FR3′ comprises an amino acid sequence selected from SEQ ID NO: 39, SEQ ID NO: 40, SEQ ID NO: 41 and SEQ ID NO: 42 or an equivalent variant thereof, and the FR4′ comprises an amino acid sequence selected from SEQ ID NO: 43, SEQ ID NO: 44 and SEQ ID NO: 45 or an equivalent variant thereof. In some embodiments, the antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof. In some embodiments, the antigen binding fragment is selected from scFv, (scFv)₂, Fab, Fab′ or F(ab′)2 of a PD-L1 antibody.

In some embodiments, the FR1 comprises an amino acid sequence as shown in SEQ ID NO: 18 or an equivalent variant thereof, the FR2 comprises an amino acid sequence as shown in SEQ ID NO: 22 or an equivalent variant thereof, the FR3 comprises an amino acid sequence as shown in SEQ ID NO: 26 or an equivalent variant thereof, the FR4 comprises an amino acid sequence as shown in SEQ ID NO: 30 or an equivalent variant thereof, the FR1′ comprises an amino acid sequence as shown in SEQ ID NO: 32 or an equivalent variant thereof, the FR2′ comprises an amino acid sequence as shown in SEQ ID NO: 35 or an equivalent variant thereof, the FR3′ comprises an amino acid sequence as shown in SEQ ID NO: 39 or an equivalent variant thereof, and the FR4′ comprises an amino acid sequence as shown in SEQ ID NO: 43 or an equivalent variant thereof.

In some embodiments, the FR1 comprises an amino acid sequence as shown in SEQ ID NO: 18 or an equivalent variant thereof, the FR2 comprises an amino acid sequence as shown in SEQ ID NO: 22 or an equivalent variant thereof, the FR3 comprises an amino acid sequence as shown in SEQ ID NO: 26 or an equivalent variant thereof, the FR4 comprises an amino acid sequence as shown in SEQ ID NO: 30 or an equivalent variant thereof, the FR1′ comprises an amino acid sequence as shown in SEQ ID NO: 32 or an equivalent variant thereof, the FR2′ comprises an amino acid sequence as shown in SEQ ID NO: 36 or an equivalent variant thereof, the FR3′ comprises an amino acid sequence as shown in SEQ ID NO: 39 or an equivalent variant thereof, and the FR4′ comprises an amino acid sequence as shown in SEQ ID NO: 43 or an equivalent variant thereof.

In some embodiments, the FR1 comprises an amino acid sequence as shown in SEQ ID NO: 19 or an equivalent variant thereof, the FR2 comprises an amino acid sequence as shown in SEQ ID NO: 23 or an equivalent variant thereof, the FR3 comprises an amino acid sequence as shown in SEQ ID NO: 27 or an equivalent variant thereof, the FR4 comprises an amino acid sequence as shown in SEQ ID NO: 30 or an equivalent variant thereof, the FR1′ comprises an amino acid sequence as shown in SEQ ID NO: 33 or an equivalent variant thereof, the FR2′ comprises an amino acid sequence as shown in SEQ ID NO: 37 or an equivalent variant thereof, the FR3′ comprises an amino acid sequence as shown in SEQ ID NO: 40 or an equivalent variant thereof, and the FR4′ comprises an amino acid sequence as shown in SEQ ID NO: 44 or an equivalent variant thereof.

In some embodiments, the FR1 comprises an amino acid sequence as shown in SEQ ID NO: 20 or an equivalent variant thereof, the FR2 comprises an amino acid sequence as shown in SEQ ID NO: 24 or an equivalent variant thereof, the FR3 comprises an amino acid sequence as shown in SEQ ID NO: 28 or an equivalent variant thereof, the FR4 comprises an amino acid sequence as shown in SEQ ID NO: 30 or an equivalent variant thereof, the FR1′ comprises an amino acid sequence as shown in SEQ ID NO: 34 or an equivalent variant thereof, the FR2′ comprises an amino acid sequence as shown in SEQ ID NO: 38 or an equivalent variant thereof, the FR3′ comprises an amino acid sequence as shown in SEQ ID NO: 41 or an equivalent variant thereof, and the FR4′ comprises an amino acid sequence as shown in SEQ ID NO: 45 or an equivalent variant thereof.

In some embodiments, the FR1 comprises an amino acid sequence as shown in SEQ ID NO: 21 or an equivalent variant thereof, the FR2 comprises an amino acid sequence as shown in SEQ ID NO: 25 or an equivalent variant thereof, the FR3 comprises an amino acid sequence as shown in SEQ ID NO: 29 or an equivalent variant thereof, the FR4 comprises an amino acid sequence as shown in SEQ ID NO: 31 or an equivalent variant thereof, the FR1′ comprises an amino acid sequence as shown in SEQ ID NO: 34 or an equivalent variant thereof, the FR2′ comprises an amino acid sequence as shown in SEQ ID NO: 35 or an equivalent variant thereof, the FR3′ comprises an amino acid sequence as shown in SEQ ID NO: 42 or an equivalent variant thereof, and the FR4′ comprises an amino acid sequence as shown in SEQ ID NO: 44 or an equivalent variant thereof.

In some embodiments, the antibody or an antigen-binding fragment thereof comprises the heavy chain variable region of an amino acid sequence as shown in SEQ ID NO: 46 or an equivalent variant thereof, and the light chain variable region of an amino acid sequence as shown in SEQ ID NO: 51 or an equivalent variant thereof.

In some embodiments, the antibody or an antigen-binding fragment thereof comprises the heavy chain variable region of an amino acid sequence as shown in SEQ ID NO: 46 or an equivalent variant thereof, and the light chain variable region of an amino acid sequence as shown in SEQ ID NO: 52 or an equivalent variant thereof.

In some embodiments, the antibody or an antigen-binding fragment thereof comprises the heavy chain variable region of an amino acid sequence as shown in SEQ ID NO: 47 or an equivalent variant thereof, and the light chain variable region of an amino acid sequence as shown in SEQ ID NO: 53 or an equivalent variant thereof.

In some embodiments, the antibody or an antigen-binding fragment thereof comprises the heavy chain variable region of an amino acid sequence as shown in SEQ ID NO: 48 or an equivalent variant thereof, and the light chain variable region of an amino acid sequence as shown in SEQ ID NO: 54 or an equivalent variant thereof.

In some embodiments, the antibody or an antigen-binding fragment thereof comprises the heavy chain variable region of an amino acid sequence as shown in SEQ ID NO: 49 or an equivalent variant thereof, and the light chain variable region of an amino acid sequence as shown in SEQ ID NO: 55 or an equivalent variant thereof.

In some embodiments, the antibody or an antigen-binding fragment thereof comprises the heavy chain variable region of an amino acid sequence as shown in SEQ ID NO: 50 or an equivalent variant thereof, and the light chain variable region of an amino acid sequence as shown in SEQ ID NO: 56 or an equivalent variant thereof.

In another aspect of the present invention, the anti-PD-L1 antibody or an antigen-binding fragment thereof of the bifunctional molecule comprises: (i) a heavy chain comprising an amino acid sequence selected from SEQ ID NO: 59, SEQ ID NO: 60, SEQ ID NO: 61, SEQ ID NO: 62 and SEQ ID NO: 63 or an equivalent fragment thereof; and (ii) a light chain comprising an amino acid sequence selected from SEQ ID NO: 64, SEQ ID NO: 65, SEQ ID NO: 66, SEQ ID NO: 67, SEQ ID NO: 68 and SEQ ID NO: 69 or an equivalent fragment thereof. In some embodiments, the antigen binding fragment is selected from scFv, (scFv)₂, Fab, Fab′ or F(ab′)2 of a PD-L1 antibody.

In some embodiments, the anti-PD-L1 antibody or an antigen-binding fragment thereof of the bifunctional molecule comprises the heavy chain of an amino acid sequence as shown in SEQ ID NO: 59 or an equivalent variant thereof, and the light chain of an amino acid sequence as shown in SEQ ID NO: 64 or an equivalent variant thereof.

In some embodiments, the anti-PD-L1 antibody or an antigen-binding fragment thereof of the bifunctional molecule comprises the heavy chain of an amino acid sequence as shown in SEQ ID NO: 59 or an equivalent variant thereof, and the light chain of an amino acid sequence as shown in SEQ ID NO: 65 or an equivalent variant thereof.

In some embodiments, the anti-PD-L1 antibody or an antigen-binding fragment thereof of the bifunctional molecule comprises the heavy chain of an amino acid sequence as shown in SEQ ID NO: 60 or an equivalent variant thereof, and the light chain of an amino acid sequence as shown in SEQ ID NO: 66 or an equivalent variant thereof.

In some embodiments, the anti-PD-L1 antibody or an antigen-binding fragment thereof of the bifunctional molecule comprises the heavy chain of an amino acid sequence as shown in SEQ ID NO: 61 or an equivalent variant thereof, and the light chain of an amino acid sequence as shown in SEQ ID NO: 67 or an equivalent variant thereof.

In some embodiments, the anti-PD-L1 antibody or an antigen-binding fragment thereof of the bifunctional molecule comprises the heavy chain of an amino acid sequence as shown in SEQ ID NO: 62 or an equivalent variant thereof, and the light chain of an amino acid sequence as shown in SEQ ID NO: 68 or an equivalent variant thereof.

In some embodiments, the anti-PD-L1 antibody or an antigen-binding fragment thereof of the bifunctional molecule comprises the heavy chain of an amino acid sequence as shown in SEQ ID NO: 63 or an equivalent variant thereof, and the light chain of an amino acid sequence as shown in SEQ ID NO: 69 or an equivalent variant thereof.

### Anti-PD-L1 Monoclonal Antibody

As used herein, the term “monoclonal antibody” refers to an antibody obtained from a substantially homogeneous antibody population, that is, except for possible natural mutants that may exist in a small amount, each antibody constituting the population is consistent. The monoclonal antibody has high specificity and can target a single antigen site. Furthermore, in contrast to a conventional (polyclonal) antibody preparation, which usually includes a plurality of different antibodies against a plurality of different determinants (epitopes), each monoclonal antibody is directed against only a single determinant on an antigen. The term “monoclonal” indicates the characteristics of an antibody obtained from a substantially homogeneous antibody population, and it cannot be understood that the antibody needs to be prepared by any specific method. For example, the monoclonal antibody used in the present invention can be prepared by a hybridoma or recombinant DNA method. The monoclonal antibody may include a “chimeric” antibody.

In an aspect of the present invention, the monoclonal antibody or an antigen-binding fragment thereof binding to human PD-L1 of the present invention comprises: (i) heavy chain complementarity determining regions CDR1, CDR2 and CDR3, wherein the CDR1 comprises an amino acid sequence as shown in SEQ ID NO: 2 or SEQ ID NO: 3 or an equivalent variant thereof, the CDR2 comprises an amino acid sequence as shown in SEQ ID NO: 5 or SEQ ID NO: 6 or an equivalent variant thereof; and the CDR3 comprises an amino acid sequence as shown in SEQ ID NO: 8 or SEQ ID NO: 9 or an equivalent variant thereof; and (ii) light chain complementarity determining regions CDR1′, CDR2′ and CDR3′, wherein the CDR1′ comprises an amino acid sequence as shown in SEQ ID NO: 11 or SEQ ID NO: 12 or an equivalent variant thereof, the CDR2′ comprises an amino acid sequence as shown in SEQ ID NO: 14 or SEQ ID NO: 15 or an equivalent variant thereof, and the CDR3′ comprises an amino acid sequence as shown in SEQ ID NO: 16 or SEQ ID NO: 17 or an equivalent variant thereof. In some embodiments, the monoclonal antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof. In some embodiments, the antigen binding fragment is selected from scFv, (scFv)₂, Fab, Fab′ or F(ab′)₂ of a PD-L1 antibody.

In some embodiments, the CDR1 comprises an amino acid sequence as shown in SEQ ID NO: 2 or an equivalent variant thereof, the CDR2 comprises an amino acid sequence as shown in SEQ ID NO: 5 or an equivalent variant thereof, the CDR3 comprises an amino acid sequence as shown in SEQ ID NO: 8 or an equivalent variant thereof, the CDR1′ comprises an amino acid sequence as shown in SEQ ID NO: 11 or an equivalent variant thereof, the CDR2′ comprises an amino acid sequence as shown in SEQ ID NO: 14 or an equivalent variant thereof, and the CDR3′ comprises an amino acid sequence as shown in SEQ ID NO: 16 or an equivalent variant thereof.

In some embodiments, the CDR1 comprises an amino acid sequence as shown in SEQ ID NO: 3 or an equivalent variant thereof, the CDR2 comprises an amino acid sequence as shown in SEQ ID NO: 6 or an equivalent variant thereof, the CDR3 comprises an amino acid sequence as shown in SEQ ID NO: 9 or an equivalent variant thereof, the CDR1′ comprises an amino acid sequence as shown in SEQ ID NO: 12 or an equivalent variant thereof, the CDR2′ comprises an amino acid sequence as shown in SEQ ID NO: 15 or an equivalent variant thereof, and the CDR3′ comprises an amino acid sequence as shown in SEQ ID NO: 17 or an equivalent variant thereof.

In another aspect, the monoclonal antibody or an antigen-binding fragment thereof binding to human PD-L1 of the present invention comprises: (i) heavy chain complementarity determining regions CDR1, CDR2 and CDR3, wherein the CDR1 comprises an amino acid sequence as shown in SEQ ID NO: 2 or SEQ ID NO: 3 or an equivalent variant thereof, the CDR2 comprises an amino acid sequence as shown in SEQ ID NO: 5 or SEQ ID NO: 6 or an equivalent variant thereof, and the CDR3 comprises an amino acid sequence as shown in SEQ ID NO: 8 or SEQ ID NO: 9 or an equivalent variant thereof; (ii) heavy chain framework regions FR1, FR2, FR3 and FR4, wherein the FR1 comprises an amino acid sequence as shown in SEQ ID NO: 18 or SEQ ID NO: 19 or an equivalent variant thereof, the FR2 comprises an amino acid sequence as shown in SEQ ID NO: 22 or SEQ ID NO: 23 or an equivalent variant thereof, the FR3 comprises an amino acid sequence as shown in SEQ ID NO: 26 or SEQ ID NO: 27 or an equivalent variant thereof, and the FR4 comprises an amino acid sequence as shown in SEQ ID NO: 30 or an equivalent variant thereof; (iii) light chain complementarity determining regions CDR1′, CDR2′ and CDR3′, wherein the CDR1′ comprises an amino acid sequence as shown in SEQ ID NO: 11 or SEQ ID NO: 12 or an equivalent variant thereof, the CDR2′ comprises an amino acid sequence as shown in SEQ ID NO: 14 or SEQ ID NO: 15 or an equivalent variant thereof, and the CDR3′ comprises an amino acid sequence as shown in SEQ ID NO: 16 or SEQ ID NO: 17 or an equivalent variant thereof; and (iv) light chain framework regions FR1′, FR2′, FR3′ and FR4′, wherein the FR1′ comprises an amino acid sequence as shown in SEQ ID NO: 32 or SEQ ID NO: 33 or an equivalent variant thereof, the FR2′ comprises an amino acid sequence as shown in SEQ ID NO: 36 or SEQ ID NO: 37 or an equivalent variant thereof, the FR3′ comprises an amino acid sequence as shown in SEQ ID NO: 39 or SEQ ID NO: 40 or an equivalent variant thereof, and the FR4′ comprises an amino acid sequence as shown in SEQ ID NO: 43 or SEQ ID NO: 44 or an equivalent variant thereof. In some embodiments, the monoclonal antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof. In some embodiments, the antigen binding fragment is selected from scFv, (scFv)₂, Fab, Fab′ or F(ab′)₂ of a PD-L1 antibody.

In some embodiments, the FR1 comprises an amino acid sequence as shown in SEQ ID NO: 18 or an equivalent variant thereof, the FR2 comprises an amino acid sequence as shown in SEQ ID NO: 22 or an equivalent variant thereof, the FR3 comprises an amino acid sequence as shown in SEQ ID NO: 26 or an equivalent variant thereof, the FR4 comprises an amino acid sequence as shown in SEQ ID NO: 30 or an equivalent variant thereof, the FR1′ comprises an amino acid sequence as shown in SEQ ID NO: 32 or an equivalent variant thereof, the FR2′ comprises an amino acid sequence as shown in SEQ ID NO: 36 or an equivalent variant thereof, the FR3′ comprises an amino acid sequence as shown in SEQ ID NO: 39 or an equivalent variant thereof, and the FR4′ comprises an amino acid sequence as shown in SEQ ID NO: 43 or an equivalent variant thereof.

In some embodiments, the FR1 comprises an amino acid sequence as shown in SEQ ID NO: 19 or an equivalent variant thereof, the FR2 comprises an amino acid sequence as shown in SEQ ID NO: 23 or an equivalent variant thereof, the FR3 comprises an amino acid sequence as shown in SEQ ID NO: 27 or an equivalent variant thereof, the FR4 comprises an amino acid sequence as shown in SEQ ID NO: 30 or an equivalent variant thereof, the FR1′ comprises an amino acid sequence as shown in SEQ ID NO: 33 or an equivalent variant thereof, the FR2′ comprises an amino acid sequence as shown in SEQ ID NO: 37 or an equivalent variant thereof, the FR3′ comprises an amino acid sequence as shown in SEQ ID NO: 40 or an equivalent variant thereof, and the FR4′ comprises an amino acid sequence as shown in SEQ ID NO: 44 or an equivalent variant thereof.

In another aspect, the monoclonal antibody or an antigen-binding fragment thereof binding to human PD-L1 of the present invention comprises: (i) a heavy chain variable region including an amino acid sequence as shown in SEQ ID NO: 47 or SEQ ID NO: 48 or an equivalent variant thereof; and (ii) a light chain variable region including an amino acid sequence as shown in SEQ ID NO: 53 or SEQ ID NO: 54 or an equivalent variant thereof. In some embodiments, the monoclonal antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof. In some embodiments, the antigen binding fragment is selected from scFv, (scFv)₂, Fab, Fab′ or F(ab′)₂ of a PD-L1 antibody.

In some embodiments, the monoclonal antibody or an antigen-binding fragment thereof binding to human PD-L1 comprises the heavy chain variable region of an amino acid sequence as shown in SEQ ID NO: 47 or an equivalent variant thereof, and the light chain variable region of an amino acid sequence as shown in SEQ ID NO: 53 or an equivalent variant thereof.

In some embodiments, the monoclonal antibody or an antigen-binding fragment thereof binding to human PD-L1 comprises the heavy chain variable region of an amino acid sequence as shown in SEQ ID NO: 48 or an equivalent variant thereof, and the light chain variable region of an amino acid sequence as shown in SEQ ID NO: 54 or an equivalent variant thereof.

In another aspect of the present invention, the monoclonal antibody or an antigen-binding fragment thereof binding to human PD-L1 of the present invention comprises: (i) a heavy chain comprising an amino acid sequence as shown in SEQ ID NO: 60 or SEQ ID NO: 61 or an equivalent variant thereof; and (ii) a light chain comprising an amino acid sequence as shown in SEQ ID NO: 66 or SEQ ID NO: 67 or an equivalent variant thereof.

In some embodiments, the monoclonal antibody or an antigen-binding fragment thereof binding to human PD-L1 comprises the heavy chain of an amino acid sequence as shown in SEQ ID NO: 60 or an equivalent variant thereof, and the light chain of an amino acid sequence as shown in SEQ ID NO: 66 or an equivalent variant thereof.

In some embodiments, the monoclonal antibody or an antigen-binding fragment thereof binding to human PD-L1 comprises the heavy chain of an amino acid sequence as shown in SEQ ID NO: 61 or an equivalent variant thereof, and the light chain of an amino acid sequence as shown in SEQ ID NO: 67 or an equivalent variant thereof.

“Specific binding” refers to that the antibody or an antigen-binding fragment thereof of the present invention can specifically interact with at least two, three, four, five, six, seven, eight or more amino acids of a target molecule. The “specific binding” of an antibody is mainly characterized by two parameters: a qualitative parameter (a binding epitope or an antibody binding position) and a quantitative parameter (binding affinity or binding strength). An antibody binding epitope can be determined by a FACS method, a peptide-spot epitope mapping, a mass spectrometry method or a peptide ELISA method. The binding strength of an antibody to a specific epitope can be determined by a Biacore method and/or an ELISA method. A signal-to-noise ratio is generally used as a representative determination and calculation method of binding specificity. In such a signal-to-noise ratio, the signal represents the intensity of an antibody binding to a target epitope, while the noise represents the intensity of the antibody binding to other non-target epitopes. Preferably, when the signal-to-noise ratio of the target epitope is about 50, it can be considered that the evaluated antibody binds to the target epitope in a specific way, i.e., “specific binding”.

An antigen-binding protein (including an antibody) “specifically binds” to an antigen if it binds to the antigen with high binding affinity as determined by a value of a dissociation constant (KD, or a corresponding Kb, as defined below). As used herein, the term “KD” refers to an equilibrium dissociation constant of a specific antibody-antigen interaction.

### Variant

As used herein, a sequence “variant” refers to a sequence that differs from the sequence as shown at one or more amino acid residues but retains the biological activity of the resulting molecule.

A “conservatively modified variant” or “conservative amino acid substitution” refers to amino acid substitution known to those skilled in the art, and making such substitution generally does not change the biological activity of the obtained molecule. In general, it is recognized by those skilled in the art that a single amino acid substitution in a non-essential region of a polypeptide substantially does not change the biological activity.

As used herein, “% identity” between two sequences refers to a function of the number of equivalent positions shared by the sequences (i.e. % homology=the number of equivalent positions/the total number of positions×100), wherein the number of gaps and the length of each gap are taken into account, and the gaps need to be introduced when the two sequences are optimally aligned. Comparison of sequences and determination of % identity between two sequences can be accomplished by mathematical algorithms. For example, the % identity between two amino acid sequences can be determined by algorithms of E. Meyers and W. Miller (Comput. Appl. Biosci., 4: 11-17 (1988)). The algorithms have yet been introduced into ALIGN program (version 2.0), which use a PAM120 weight residue table, with a Gap Length Penalty of 12 and a Gap Penalty of 4. Furthermore, the % identity between two amino acid sequences can be determined by algorithms of Needleman and Wunsch (J. MoI. Biol. 48: 444-453 (1970)). The algorithms have yet been introduced into GAP program of a GCG software package (available at www.gcg.com), which use a Blossum 62 matrix or a PAM250 matrix, with a gap weight of 16, 14, 12, 10, 8, 6 or 4, and a length weight of 1, 2, 3, 4, 5 or 6.

When referring to a ligand/receptor, an antibody/antigen or other binding pairs, “specific” binding refers to determining whether there is a binding reaction of the protein in a heterogeneous population of proteins and/or other biochemical reagents. Therefore, under the specified conditions, a specific ligand/antigen binds to a specific receptor/antibody, and does not bind to other proteins present in a sample in a significant amount.

An “equivalent variant” refers to a sequence that is identical or substantially similar to the sequence as shown (e.g, an amino acid sequence) in biological activity and function, but has about 80%, about 85%, about 90%, about 95%, about 96%, about 97%, about 98% or about 99% identity with the sequence as shown.

### Antibody Purification

When recombinant technology is used, an antibody can be produced in a cell or a periplasmic space, or secreted directly into a culture medium. If the antibody is produced in a cell, then as a first step, particle fragments (host cells or lysed fragments) are removed, for example, by centrifugation or ultrafiltration. When the antibody is secreted into a culture medium, the supernatant from the expression system is usually first concentrated with a commercially-available protein concentration filter (e.g., an Amicon or Millipore Pellicon ultrafiltration unit). A protease inhibitor (e.g., PMSF) can be used in any of the foregoing steps to inhibit proteolysis, and antibiotics can be used to prevent the growth of foreign pollutants.

Depending on the antibody to be recovered, other protein purification techniques can also be used, for example fractionation on an ion exchange column, ethanol precipitation, reversed-phase HPLC, silica gel chromatography, an anion or cation exchange resin (e.g. a polyaspartic acid column) chromatography, chromatofocusing, SDS-PAGE and ammonium sulfate precipitation. In an embodiment, a glycoprotein can be purified by the following methods: adsorbing the glycoprotein onto a lectin substrate (e.g. a lectin affinity column) to remove the fucose-containing glycoprotein from the preparation and thereby enrich the fucose-free glycoprotein.

### Linker

A C-terminal of the antibody or an antigen-binding fragment thereof in the bifunctional molecule of the present invention can be connected with an N-terminal of the human TGFβRII or a functional fragment thereof capable of binding to human TGFβ through a linker. The linker can be any linker known in the art. In some embodiments, the amino acid sequence of the linker is GGGGSGGGGSGGGGSGGGGS (SEQ ID NO: 70).

### Bifunctional Molecule Binding to Human TGFβ and PD-L1

In an aspect, the bifunctional molecule binding to human TGFβ beta and PD-L1 of the present invention comprises: (a) human TGFβRII or a fragment thereof capable of binding to human TGFβ, and (b) an antibody or an antigen-binding fragment thereof binding to human PD-L1, wherein the antibody or the antigen-binding fragment thereof comprises: (i) heavy chain complementarity determining regions CDR1, CDR2 and CDR3, wherein the CDR1 comprises an amino acid sequence selected from SEQ ID NO: 1, SEQ ID NO: 2 and SEQ ID NO: 3 or an equivalent variant thereof, the CDR2 comprises an amino acid sequence selected from SEQ ID NO: 4, SEQ ID NO: 5 and SEQ ID NO: 6 or an equivalent variant thereof, and the CDR3 comprises an amino acid sequence selected from SEQ ID NO: 7, SEQ ID NO: 8 and SEQ ID NO: 9 or an equivalent variant thereof; and (ii) light chain complementarity determining regions CDR1′, CDR2′ and CDR3′, wherein the CDR1′ comprises an amino acid sequence selected from SEQ ID NO: 10, SEQ ID NO: 11 and SEQ ID NO: 12 or an equivalent variant thereof, the CDR2′ comprises an amino acid sequence selected from SEQ ID NO: 13, SEQ ID NO: 14 and SEQ ID NO: 15 or an equivalent variant thereof, and the CDR3′ comprises an amino acid sequence as shown in SEQ ID NO: 16 or SEQ ID NO: 17 or an equivalent variant thereof. In some embodiments, the antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof. In some embodiments, the antigen binding fragment is selected from scFv, (scFv)₂, Fab, Fab′ or F(ab′)₂ of a PD-L1 antibody. In some embodiments, a C-terminal of the antibody or the antigen-binding fragment thereof is connected with an N-terminal of the human TGFβRII or a functional fragment thereof capable of binding to human TGFβ through a linker. In some embodiments, an amino acid sequence of the linker is as shown in SEQ ID NO: 70. In some embodiments, an amino acid sequence of the human TGFβRII is as shown in SEQ ID NO: 71. In some embodiments, the bifunctional molecule has a three-dimensional spatial structure.

In some embodiments, the CDR1 comprises an amino acid sequence as shown in SEQ ID NO: 1 or an equivalent variant thereof, the CDR2 comprises an amino acid sequence as shown in SEQ ID NO: 4 or an equivalent variant thereof, the CDR3 comprises an amino acid sequence as shown in SEQ ID NO: 7 or an equivalent variant thereof, the CDR1′ comprises an amino acid sequence as shown in SEQ ID NO: 10 or an equivalent variant thereof, the CDR2′ comprises an amino acid sequence as shown in SEQ ID NO: 13 or an equivalent variant thereof, and the CDR3′ comprises an amino acid sequence as shown in SEQ ID NO: 16 or an equivalent variant thereof.

In some embodiments, the CDR1 comprises an amino acid sequence as shown in SEQ ID NO: 2 or an equivalent variant thereof, the CDR2 comprises an amino acid sequence as shown in SEQ ID NO: 5 or an equivalent variant thereof, the CDR3 comprises an amino acid sequence as shown in SEQ ID NO: 8 or an equivalent variant thereof, the CDR1′ comprises an amino acid sequence as shown in SEQ ID NO: 11 or an equivalent variant thereof, the CDR2′ comprises an amino acid sequence as shown in SEQ ID NO: 14 or an equivalent variant thereof, and the CDR3′ comprises an amino acid sequence as shown in SEQ ID NO: 16 or an equivalent variant thereof.

In some embodiments, the CDR1 comprises an amino acid sequence as shown in SEQ ID NO: 3 or an equivalent variant thereof, the CDR2 comprises an amino acid sequence as shown in SEQ ID NO: 6 or an equivalent variant thereof, the CDR3 comprises an amino acid sequence as shown in SEQ ID NO: 9 or an equivalent variant thereof, the CDR1′ comprises an amino acid sequence as shown in SEQ ID NO: 12 or an equivalent variant thereof, the CDR2′ comprises an amino acid sequence as shown in SEQ ID NO: 15 or an equivalent variant thereof, and the CDR3′ comprises an amino acid sequence as shown in SEQ ID NO: 19 or an equivalent variant thereof.

In another aspect, the bifunctional molecule binding to human TGFβ beta and PD-L1 of the present invention comprises: (a) human TGFβRII or a functional fragment thereof capable of binding to human TGFβ, and (b) an antibody or an antigen-binding fragment thereof binding to human PD-L1, wherein the antibody or the antigen-binding fragment thereof comprises: (i) heavy chain complementarity determining regions CDR1, CDR2 and CDR3, wherein the CDR1 comprises an amino acid sequence selected from SEQ ID NO: 1, SEQ ID NO: 2 and SEQ ID NO: 3 or an equivalent variant thereof, the CDR2 comprises an amino acid sequence selected from SEQ ID NO: 4, SEQ ID NO: 5 and SEQ ID NO: 6 or an equivalent variant thereof, and the CDR3 comprises an amino acid sequence selected from SEQ ID NO: 7, SEQ ID NO: 8 and SEQ ID NO: 9 or an equivalent variant thereof; (ii) heavy chain framework regions FR1, FR2, FR3 and FR4, wherein the FR1 comprises an amino acid sequence selected from SEQ ID NO: 18, SEQ ID NO: 19, SEQ ID NO: 20 and SEQ ID NO: 21 or an equivalent variant thereof, the FR2 comprises an amino acid sequence selected from SEQ ID NO: 22, SEQ ID NO: 23, SEQ ID NO: 24 and SEQ ID NO: 25 or an equivalent variant thereof, the FR3 comprises an amino acid sequence selected from SEQ ID NO: 26, SEQ ID NO: 27, SEQ ID NO: 28 and SEQ ID NO: 29 or an equivalent variant thereof, and the FR4 comprises an amino acid sequence as shown in SEQ ID NO: 30 or SEQ ID NO: 31 or an equivalent variant thereof; (iii) light chain complementarity determining regions CDR1′, CDR2′ and CDR3′, wherein the CDR1′ comprises an amino acid sequence selected from SEQ ID NO: 10, SEQ ID NO: 11 and SEQ ID NO: 12 or an equivalent variant thereof, the CDR2′ comprises an amino acid sequence selected from SEQ ID NO: 13, SEQ ID NO: 14 and SEQ ID NO: 15 or an equivalent variant thereof, and the CDR3′ comprises an amino acid sequence as shown in SEQ ID NO: 16 or SEQ ID NO: 17 or an equivalent variant thereof; and (iv) light chain framework regions FR1′, FR2′, FR3′ and FR4′, wherein the FR1′ comprises an amino acid sequence selected from SEQ ID NO: 32, SEQ ID NO: 33 and SEQ ID NO: 34 or an equivalent variant thereof, the FR2′ comprises an amino acid sequence selected from SEQ ID NO: 35, SEQ ID NO: 36, SEQ ID NO: 37 and SEQ ID NO: 38 or an equivalent variant thereof, the FR3′ comprises an amino acid sequence selected from SEQ ID NO: 39, SEQ ID NO: 40, SEQ ID NO: 41 and SEQ ID NO: 42 or an equivalent variant thereof, and the FR4′ comprises an amino acid sequence selected from SEQ ID NO: 43, SEQ ID NO: 44 and SEQ ID NO: 45 or an equivalent variant thereof. In some embodiments, the antibody or the antigen-binding fragment thereof comprises a heavy chain constant region and a light chain constant region, wherein the heavy chain constant region comprises an amino acid sequence as shown in SEQ ID NO: 57 or an equivalent variant thereof, and the light chain constant region comprises an amino acid sequence as shown in SEQ ID NO: 58 or an equivalent variant thereof. In some embodiments, the antigen binding fragment is selected from scFv, (scFv)₂, Fab, Fab′ or F(ab′)₂ of a PD-L1 antibody. In some embodiments, a C-terminal of the antibody or the antigen-binding fragment thereof is connected with an N-terminal of the human TGFβRII or a functional fragment thereof capable of binding to human TGFβ through a linker. In some embodiments, an amino acid sequence of the linker is as shown in SEQ ID NO: 70. In some embodiments, an amino acid sequence of the human TGFβRII is as shown in SEQ ID NO: 71. In some embodiments, the bifunctional molecule has a three-dimensional spatial structure.

In some embodiments, the FR1 comprises an amino acid sequence as shown in SEQ ID NO: 18 or an equivalent variant thereof, the FR2 comprises an amino acid sequence as shown in SEQ ID NO: 22 or an equivalent variant thereof, the FR3 comprises an amino acid sequence as shown in SEQ ID NO: 26 or an equivalent variant thereof, the FR4 comprises an amino acid sequence as shown in SEQ ID NO: 30 or an equivalent variant thereof, the FR1′ comprises an amino acid sequence as shown in SEQ ID NO: 32 or an equivalent variant thereof, the FR2′ comprises an amino acid sequence as shown in SEQ ID NO: 35 or an equivalent variant thereof, the FR3′ comprises an amino acid sequence as shown in SEQ ID NO: 39 or an equivalent variant thereof, and the FR4′ comprises an amino acid sequence as shown in SEQ ID NO: 43 or an equivalent variant thereof.

In some embodiments, the FR1 comprises an amino acid sequence as shown in SEQ ID NO: 18 or an equivalent variant thereof, the FR2 comprises an amino acid sequence as shown in SEQ ID NO: 22 or an equivalent variant thereof, the FR3 comprises an amino acid sequence as shown in SEQ ID NO: 26 or an equivalent variant thereof, the FR4 comprises an amino acid sequence as shown in SEQ ID NO: 30 or an equivalent variant thereof, the FR1′ comprises an amino acid sequence as shown in SEQ ID NO: 32 or an equivalent variant thereof, the FR2′ comprises an amino acid sequence as shown in SEQ ID NO: 36 or an equivalent variant thereof, the FR3′ comprises an amino acid sequence as shown in SEQ ID NO: 39 or an equivalent variant thereof, and the FR4′ comprises an amino acid sequence as shown in SEQ ID NO: 43 or an equivalent variant thereof.

In some embodiments, the FR1 comprises an amino acid sequence as shown in SEQ ID NO: 19 or an equivalent variant thereof, the FR2 comprises an amino acid sequence as shown in SEQ ID NO: 23 or an equivalent variant thereof, the FR3 comprises an amino acid sequence as shown in SEQ ID NO: 27 or an equivalent variant thereof, the FR4 comprises an amino acid sequence as shown in SEQ ID NO: 30 or an equivalent variant thereof, the FR1′ comprises an amino acid sequence as shown in SEQ ID NO: 33 or an equivalent variant thereof, the FR2′ comprises an amino acid sequence as shown in SEQ ID NO: 37 or an equivalent variant thereof, the FR3′ comprises an amino acid sequence as shown in SEQ ID NO: 40 or an equivalent variant thereof, and the FR4′ comprises an amino acid sequence as shown in SEQ ID NO: 44 or an equivalent variant thereof.

In some embodiments, the FR1 comprises an amino acid sequence as shown in SEQ ID NO: 20 or an equivalent variant thereof, the FR2 comprises an amino acid sequence as shown in SEQ ID NO: 24 or an equivalent variant thereof, the FR3 comprises an amino acid sequence as shown in SEQ ID NO: 28 or an equivalent variant thereof, the FR4 comprises an amino acid sequence as shown in SEQ ID NO: 30 or an equivalent variant thereof, the FR1′ comprises an amino acid sequence as shown in SEQ ID NO: 34 or an equivalent variant thereof, the FR2′ comprises an amino acid sequence as shown in SEQ ID NO: 38 or an equivalent variant thereof, the FR3′ comprises an amino acid sequence as shown in SEQ ID NO: 41 or an equivalent variant thereof, and the FR4′ comprises an amino acid sequence as shown in SEQ ID NO: 45 or an equivalent variant thereof.

In some embodiments, the FR1 comprises an amino acid sequence as shown in SEQ ID NO: 21 or an equivalent variant thereof, the FR2 comprises an amino acid sequence as shown in SEQ ID NO: 25 or an equivalent variant thereof, the FR3 comprises an amino acid sequence as shown in SEQ ID NO: 29 or an equivalent variant thereof, the FR4 comprises an amino acid sequence as shown in SEQ ID NO: 31 or an equivalent variant thereof, the FR1′ comprises an amino acid sequence as shown in SEQ ID NO: 34 or an equivalent variant thereof, the FR2′ comprises an amino acid sequence as shown in SEQ ID NO: 35 or an equivalent variant thereof, the FR3′ comprises an amino acid sequence as shown in SEQ ID NO: 42 or an equivalent variant thereof, and the FR4′ comprises an amino acid sequence as shown in SEQ ID NO: 44 or an equivalent variant thereof.

In some embodiments, the antibody or an antigen-binding fragment thereof included in the bifunctional molecule binding to human TGFβ-β and PD-L1 comprises the heavy chain variable region of an amino acid sequence as shown in SEQ ID NO: 46 or an equivalent variant thereof, and the light chain variable region of an amino acid sequence as shown in SEQ ID NO: 51 or an equivalent variant thereof.

In some embodiments, the antibody or an antigen-binding fragment thereof included in the bifunctional molecule binding to human TGFβ-β and PD-L1 comprises the heavy chain variable region of an amino acid sequence as shown in SEQ ID NO: 46 or an equivalent variant thereof, and the light chain variable region of an amino acid sequence as shown in SEQ ID NO: 52 or an equivalent variant thereof.

In some embodiments, the antibody or an antigen-binding fragment thereof included in the bifunctional molecule binding to human TGFβ-β and PD-L1 comprises the heavy chain variable region of an amino acid sequence as shown in SEQ ID NO: 47 or an equivalent variant thereof, and the light chain variable region of an amino acid sequence as shown in SEQ ID NO: 53 or an equivalent variant thereof.

In some embodiments, the antibody or an antigen-binding fragment thereof included in the bifunctional molecule binding human TGFβ-β and PD-L1 comprises the heavy chain variable region of an amino acid sequence as shown in SEQ ID NO: 48 or an equivalent variant thereof, and the light chain variable region of an amino acid sequence as shown in SEQ ID NO: 54 or an equivalent variant thereof.

In some embodiments, the antibody or an antigen-binding fragment thereof included in the bifunctional molecule binding human TGFβ-β and PD-L1 comprises the heavy chain variable region of an amino acid sequence as shown in SEQ ID NO: 49 or an equivalent variant thereof, and the light chain variable region of an amino acid sequence as shown in SEQ ID NO: 55 or an equivalent variant thereof.

In some embodiments, the antibody or an antigen-binding fragment thereof included in the bifunctional molecule binding to human TGFβ-β and PD-L1 comprises the heavy chain variable region of an amino acid sequence as shown in SEQ ID NO: 50 or an equivalent variant thereof, and the light chain variable region of an amino acid sequence as shown in SEQ ID NO: 56 or an equivalent variant thereof.

In another aspect, the bifunctional molecule binding to human TGFβ beta and PD-L1 of the present invention comprises: (a) human TGFβRII or a functional fragment thereof capable of binding human TGFβ, and (b) an antibody or an antigen-binding fragment thereof binding to human PD-L1, wherein the antibody or the antigen-binding fragment thereof comprises a heavy chain and a light chain, wherein the heavy chain comprises an amino acid sequence selected from SEQ ID NO: 59, SEQ ID NO: 60, SEQ ID NO: 61, SEQ ID NO: 62 and SEQ ID NO: 63 or an equivalent variant thereof, and the light chain comprises an amino acid sequence selected from SEQ ID NO: 64, SEQ ID NO: 65, SEQ ID NO: 66, SEQ ID NO: 67, SEQ ID NO: 68 and SEQ ID NO: 69 or an equivalent variant thereof. In some embodiments, the antigen binding fragment is selected from scFv, (scFv)₂, Fab, Fab′ or F(ab′)₂ of a PD-L1 antibody. In some embodiments, a C-terminal of the antibody or the antigen-binding fragment thereof is connected with an N-terminal of the human TGFβRII or a functional fragment thereof capable of binding to human TGFβ through a linker. In some embodiments, an amino acid sequence of the linker is as shown in SEQ ID NO: 70. In some embodiments, an amino acid sequence of the human TGFβRII is as shown in SEQ ID NO: 71. In some embodiments, the bifunctional molecule has a three-dimensional spatial structure.

In some embodiments, the antibody or an antigen-binding fragment thereof included in the bifunctional molecule binding to human TGFβ-β and PD-L1 comprises the heavy chain of an amino acid sequence as shown in SEQ ID NO: 59 or an equivalent variant thereof, and the light chain of an amino acid sequence as shown in SEQ ID NO: 64 or an equivalent variant thereof.

In some embodiments, the antibody or an antigen-binding fragment thereof included in the bifunctional molecule binding to human TGFβ-β and PD-L1 comprises the heavy chain of an amino acid sequence as shown in SEQ ID NO: 59 or an equivalent variant thereof, and the light chain of an amino acid sequence as shown in SEQ ID NO: 65 or an equivalent variant thereof.

In some embodiments, the antibody or an antigen-binding fragment thereof included in the bifunctional molecule binding to human TGFβ-3 and PD-L1 comprises the heavy chain of an amino acid sequence as shown in SEQ ID NO: 60 or an equivalent variant thereof, and the light chain of an amino acid sequence as shown in SEQ ID NO: 66 or an equivalent variant thereof.

In some embodiments, the antibody or an antigen-binding fragment thereof included in the bifunctional molecule binding to human TGFβ-β and PD-L1 comprises the heavy chain of an amino acid sequence as shown in SEQ ID NO: 61 or an equivalent variant thereof, and the light chain of an amino acid sequence as shown in SEQ ID NO: 67 or an equivalent variant thereof.

In some embodiments, the antibody or an antigen-binding fragment thereof included in the bifunctional molecule binding to human TGFβ-β and PD-L1 comprises the heavy chain of an amino acid sequence as shown in SEQ ID NO: 62 or an equivalent variant thereof, and the light chain of an amino acid sequence as shown in SEQ ID NO: 68 or an equivalent variant thereof.

In some embodiments, the antibody or an antigen-binding fragment thereof included in the bifunctional molecule binding to human TGFβ-β and PD-L1 comprises the heavy chain of an amino acid sequence as shown in SEQ ID NO: 63 or an equivalent variant thereof, and the light chain of an amino acid sequence as shown in SEQ ID NO: 69 or an equivalent variant thereof.

### Pharmaceutical Composition

A “pharmaceutical composition” refers to a pharmaceutical formulation for human use. The pharmaceutical composition comprises a suitable formulation of the bifunctional molecule binding to human TGFβ and PD-L1 or a monoclonal antibody or an antigen-binding fragment thereof binding to human PD-L1 of the present invention, and a carrier, a stabilizer and/or an excipient. The present invention provides a pharmaceutical formulation comprising the bifunctional molecule or monoclonal antibody or an antigen-binding fragment thereof of the present invention. In order to prepare the pharmaceutical composition or an aseptic composition, the bifunctional molecule or the antibody or an antigen-binding fragment thereof is mixed with a pharmaceutically-acceptable carrier or excipient. A formulation of therapeutic and diagnostic drugs in a form of, for example, freeze-dried powder, slurry, aqueous solution or suspension can be prepared by mixing with a physiologically acceptable carrier, excipient or stabilizer.

The toxicity and therapeutic efficacy of a composition administered alone or in combination with an immunosuppressant can be determined in a cell culture or an experimental animal by a standard pharmaceutical method, for example a method for determining LD50 (a dose that causes death of 50% of a population) and ED50 (a dose that effectively treats 50% of a population). The dose ratio of toxicity to therapeutic effect is therapeutic index, which can be expressed as the ratio of LD50 to ED50. The data obtained from these cell culture assays and animal studies can be used to adjust the dosage range for human. The dosage of the compound is preferably within a circulating concentration range of ED50 including little or no toxicity. The dosage can be varied within this range according to the adopted dosage form and the used route of administration.

Suitable routes of administration include parenteral administration (e.g., intramuscular, intravenous or subcutaneous administration) and oral administration. The antibody used in the pharmaceutical composition or for practicing the method of the present invention can be administered in a variety of conventional ways, for example via oral ingestion, inhalation, topical administration or percutaneous, subcutaneous, intraperitoneal, parenteral, intraarterial or intravenous injection. In an embodiment, the binding compound of the present invention is administered intravenously. In another embodiment, the binding compound of the present invention is administered subcutaneously. Alternatively, the antibody can be administrated in a local rather than systemic manner (usually a long-acting formulation or a sustained-release formulation), for example, by injecting the antibody directly into the site of action. Moreover, the antibody can be administrated in a targeted drug delivery system.

An appropriate dosage is determined by a clinician, for example, using a parameter or factor known or suspected to affect the treatment or expected to affect the treatment in the art. Typically, the initial dose is slightly lower than the optimal dose, and then it is increased slightly until the desired or optimal acting effect relative to any adverse side effect is achieved. Important diagnostic measurements include measuring, for example, an inflammatory symptom or a level of inflammatory cytokines as produced.

The antibody, antibody fragment and cytokine can be provided by continuous infusion or by administration at certain intervals (e.g., per day, per week, or 1-7 times per week). The dosage can be provided intravenously, subcutaneously, intraperitoneally, percutaneously, topically, orally, nasally, rectally, intramuscularly, intracerebrally, intravertebrally or by inhalation. A preferred dosage regimen is one that includes the maximum dosage or frequency of administration to avoid a significantly undesirable side effect. A total weekly dose is generally at least 0.05 μg/kg body weight, more generally at least 0.2 μg/kg, most generally at least 0.5 μg/kg, typically at least 1 μg/kg, more typically at least 10 μg/kg, most typically at least 109 μg/kg, preferably at least 0.2 mg/kg, more preferably at least 1.0 mg/kg, most preferably at least 2.0 mg/kg, ideally at least 10 mg/kg, more ideally at least 25 mg/kg and most ideally at least 50 mg/kg. On a molar/kg basis, the desired dose of a small molecular therapeutic agent, e.g., a peptide mimetic, a natural product or an organic chemical agent, is nearly the same as that of the antibody or polypeptide.

The pharmaceutical composition of the present invention may further contain other agents, including but not limited to cytotoxic agents, cell growth inhibitors, antiangiogenic drugs or antimetabolites, tumor-targeting drugs, immunostimulants or immunomodulators or antibodies conjugated with the cytotoxic agents, cell growth inhibitors or other toxic drugs. The pharmaceutical composition can also be administered together with other forms of treatment (e.g., surgery, chemotherapy and radiation). Typical veterinary, experimental or research objects include monkeys, dogs, cats, rats, mice, rabbits, guinea pigs, horses and human.

Specifically, the bifunctional molecule binding to human TGFβ and PD-L1 or monoclonal antibody or an antigen-binding fragment thereof binding to human PD-L1 of the present invention can be used in combination with a second therapeutic agent. In a specific embodiment, the second therapeutic agent and the bifunctional molecule binding to human TGFβ and PD-L1 or monoclonal antibody or an antigen-binding fragment thereof of the present invention are administered at substantially the same time. An individual sometimes uses the second therapeutic agent and the bifunctional molecule binding to human TGFβ and PD-L1 or monoclonal antibody or an antigen-binding fragment thereof simultaneously. In an embodiment, the second therapeutic agent or other agents typically administered to cancer patients and the bifunctional molecule binding to human TGFβ and PD-L1 or monoclonal antibody or an antigen-binding fragment thereof of the present invention can be combined into a pharmaceutical composition; and in other embodiments, they are administered separately.

The term “second therapeutic agent” is any agent that is advantageously combined with an anti-PD-L1 antibody. Exemplary agents that can be advantageously combined with the anti-PD-L1 antibody include, but are not limited to, other agents that inhibit PD-L1 activity (including other antibodies or antigen-binding fragments thereof, peptide inhibitors, small molecule antagonists, etc.) and/or agents that interfere with upstream or downstream signal transduction of PD-L1. The “second therapeutic agent” may also be any agent that advantageously binds to human TGFβ.

An aspect of the present invention provides a method for treating a cancer with the bifunctional molecule binding to human TGFβ and PD-L1 or monoclonal antibody or an antigen-binding fragment thereof binding to human PD-L1 of the present invention. More specifically, the present invention provides a method for enhancing the function of T cells, restoring an anti-tumor response and controlling tumor growth, which comprises administering a therapeutically effective amount of any of the bifunctional molecule binding to human TGFβ and PD-L1 or the monoclonal antibody or an antigen-binding fragment thereof binding to human PD-L1 or the pharmaceutical composition described above.

Enhancing the function of T cells, restoring the anti-tumor response and controlling tumor growth provide a broad-spectrum method for treating cancer. Therefore, various types of cancers can be treated by administering the bifunctional molecule binding to human TGFβ and PD-L1 or the monoclonal antibody or an antigen-binding fragment thereof binding to human PD-L1 or the pharmaceutical composition of the present invention.

### Treatment

When “administering” and “treating” are used with reference to an animal, human, an experimental object, a cell, a tissue, an organ or a biological fluid, it refers to contacting an exogenous drug, a therapeutic agent, a diagnostic agent or a composition with the animal, human, subject, cell, tissue, organs or biological fluid. “Administering” and “treating” can refer to, for example, therapeutic methods, pharmacokinetic methods, diagnostic methods, research methods and experimental methods. Treating cells comprises contacting a reagent with the cells and contacting a reagent with a fluid, wherein the fluid is in contact with the cells. “Administering” and “treating” also refer to in vitro and ex vivo treatment of cells, for example, by reagents, diagnostic agents, binding compositions or by other cells.

As used herein, the term “inhibiting” or “treating” includes delaying the development of symptoms associated with a disease and/or alleviating the severity of these symptoms that will or is expected to develop for the disease. The term also includes relieving existing symptoms, preventing other symptoms, and relieving or preventing the potential causes of these symptoms. Therefore, the term indicates that beneficial results have been given to vertebrate subjects suffering from diseases.

### Therapeutically Effective Amount

As used herein, the term “therapeutically effective amount” or “effective amount” refers to an amount the bifunctional molecule binding to human TGFβ and PD-L1 or the monoclonal antibody or an antigen-binding fragment thereof binding to human PD-L1 of the present invention that effectively prevents or relieves a disease or disorder to be treated when the bifunctional molecule or the monoclonal antibody or an antigen-binding fragment thereof is given to a cell, an organism or a subject alone or in combination with another therapeutic agent. A therapeutically effective amount further refers to an amount of the compound sufficient to cause symptoms to be relieved, for example treating, curing, preventing or relieving related medical conditions, or improving the treatment rate, cure rate, prevention rate or relieving rate of the symptoms. When an active ingredient is administered to an individual alone, a therapeutically effective amount refers to the amount of the ingredient alone. When a combination is administered, a therapeutically effective amount refers to the combined amount of active ingredients that produce a therapeutic effect, regardless of whether they are administered jointly, continuously or simultaneously. A therapeutically effective amount will generally relieve the symptoms by at least 10%; generally at least 20%; preferably at least about 30%; more preferably at least 40%, and most preferably at least 50%.

In the present invention, “about” refers to that a numerical value is within an acceptable error range of a specific value determined by those of ordinary skills in the art, and the numerical value depends in part on how it is measured or determined (i.e., the limit of a measurement system). For example, “about” can mean a standard deviation within or exceeding 1 in every implementation in the art. Alternatively, “about” or “substantially containing” may mean a range of up to 20%. Furthermore, especially for a biological system or process, the term can mean at most one order of magnitude or at most 5 times the numerical value. Unless otherwise specified, when a specific value appears in the present application and claims, the meaning of “about” or “substantially containing” should be assumed to be within the acceptable error range of the specific value.

### Cancer

The bifunctional molecule binding to human TGFβ and PD-L1 or the monoclonal antibody or an antigen-binding fragment thereof binding to human PD-L1 of the present invention can be used for treating cancer. The cancer comprises, but is not limited to: gastric cancer, lung cancer, liver cancer, intrahepatic cholangiocarcinoma, colon cancer, prostate cancer, pancreatic cancer, ovarian cancer, glioma, renal cancer, urothelial cell cancer, breast cancer, cervical cancer, head and neck squamous cell carcinoma, nasopharyngeal cancer, esophageal cancer, bladder cancer, renal cell carcinoma, thyroid cancer, skin cancer and oral squamous cell carcinoma. In some embodiments, the cancer is a highly immunogenic cancer. In some embodiments, the cancer is a cancer expressing increased levels of PD-L1 and/or TGFβ.

### EXAMPLES

### Example 1: Construction and Expression of Bifunctional Molecule

Preparation of transfected cells: HEK-293 cells cultured in suspension were taken to determine their cell density and survival rate. In order to ensure the transfection effect, a suspension of cells grown at an exponential phase (with a density of about 6-8×10⁶ cells/milliliter) and having a survival rate greater than 97% was used. Without centrifugation, the cells were directly added into a KOP293 culture medium (yellow and transparent, containing F-68) to make its density 3×10⁶ cells/milliliter. The shake flask was placed in a constant-temperature shaker with 5% CO₂, and cultured under shaking at a constant temperature of 37° C. and 120 rpm for 24 hours (5-6×10⁶ cells/milliliter).

Transient transfection (using the transfection system available from Zhuhai Kairui Biotech, Ltd., and taking transfecting 100 ml of the cell suspension as an example): Two sterile centrifuge tubes with a volume of 15 ml were prepared. Into one of the centrifuge tubes added was 5 ml of KRM and 100 μg of plasmids (obtained according to standard genetic engineering operation, each 50 μg of the heavy chain and the light chain) and pipetted up and down gently to mix well. Another centrifuge tube was taken, added with 5 ml of KRM and 500 μg of a PEI (a 1 μg/μl aqueous solution) transfection reagent, and pipetted up and down gently to mix well (mass ratio: plasmid:PEI=1:5). All the liquid in the centrifuge tube containing the transfection reagent was pipetted and mixed with the liquid in the centrifuge tube containing the plasmid, pipetted up and down gently to mix well, and allowed to stand at room temperature for 10 minutes. The prepared HEK-293 cells were taken out from the constant-temperature shaker, and the plasmid-vector complex was evenly added into the cell suspension, shaken evenly and put back into the CO₂ constant-temperature shaker for culturing (37° C., 120 rpm). After transfection for 3 hours, appropriate amount of antibiotics (100×P/S) could be added as needed.

Product expression and detection: After 24 hours of transfection, an expression enhancer (Kairui Protein Expression Enhancer, KPEEn) could be added to a final concentration of 3 mmol/L to increase the expression amount of the protein. Three days after transfection, the expression of the product was determined. The transfection time was 6 days (the cell viability was less than 70%). The supernatant was purified by a Protein A affinity column SDS-PAGE (as shown in FIG. 1) was run and Coomassie brilliant blue staining was conducted to analyze the results.

The purified product was sequenced, and the sequencing results were as shown in the following table.

TABLE 1
Sequences of Bifunctional Molecules

| Name of | Sequence (heavy chain (containing |
|---|---|
| bifunctional | a TGFβRII receptor domain) and |
| molecule | light chain) |
| BJ-005-P1 | EVQLVQSGAEVKKPGSSVKVSCAAS |
| | GFNITDTYMHWVRQAPGQGLEWIGW |
| | IDPANGHTKSDPKFQGKATITADTS |
| | TNTAYMELSSLRSEDTAVYYCARRG |
| | DGYRYFYAMGYWGQGTLVTVSSAST |
| | KGPSVFPLAPSSKSTSGGTAALGCL |
| | VKDYFPEPVTVSWNSGALTSGVHTF |
| | PAVLQSSGLYSLSSVVTVPSSSLGT |
| | QTYICNVNHKPSNTKVDKRVEPKSC |
| | DKTHTCPPCPAPELLGGPSVFLFPP |
| | KPKDTLMISRTPEVTCVVVDVSHED |
| | PEVKFNWYVDGVEVHNAKTKPREEQ |
| | YNSTYRVVSVLTVLHQDWLNGKEYK |
| | CKVSNKALPAPIEKTISKAKGQPRE |
| | PQVYTLPPSRDELTKNQVSLTCLVK |
| | GFYPSDIAVEWESNGQPENNYKTTP |
| | PVLDSDGSFFLYSKLTVDKSRWQQG |
| | NVFSCSVMHEALHNHYTQKSLSLSP |
| | KGGGGSGGGGSGGGGSGGGGSTIPP |
| | HVQKSVNNDMIVTDNNGAVKFPQLC |
| | KFCDVRESTCDNQKSCMSNCSITSI |
| | CEKPQEVCVAVWRKNDENITLETVC |
| | HDPKLPYHDFILEDAASPKCIMKEK |
| | KKPGETFFMCSCSSDECNDNIIFSE |
| | EYNTSNPD |
| | (SEQ ID NO: 74) |
| | DIQMTQSPSTLSASVGDRVTITCRA |
| | SQDISNYLNWYQQKPGKAPKLLIYY |
| | TSRLRSGVPARFSGSGSGTEYTLTI |
| | SSLQPDDFATYYCQQGNTLPPTFGQ |
| | GTKVEVKRTVAAPSVFIFPPSDEQL |
| | KSGTASVVCLLNNFYPREAKVQWKV |
| | DNALQSGNSQESVTEQDSKDSTYSL |
| | SSTLTLSKADYEKHKVYACEVTHQG |
| | LSSPVTKSFNRGEC |
| | (SEQ ID NO: 64) |
| BJ-005-P2 | EVQLVQSGAEVKKPGSSVKVSCAAS |
| | GFNITDTYMHWVRQAPGQGLEWIGW |
| | IDPANGHTKSDPKFQGKATITADTS |
| | TNTAYMELSSLRSEDTAVYYCARRG |
| | DGYRYFYAMGYWGQGTLVTVSSAST |
| | KGPSVFPLAPSSKSTSGGTAALGCL |
| | VKDYFPEPVTVSWNSGALTSGVHTF |
| | PAVLQSSGLYSLSSVVTVPSSSLGT |
| | QTYICNVNHKPSNTKVDKRVEPKSC |
| | DKTHTCPPCPAPELLGGPSVFLFPP |
| | KPKDTLMISRTPEVTCVVVDVSHED |
| | PEVKFNWYVDGVEVHNAKTKPREEQ |
| | YNSTYRVVSVLTVLHQDWLNGKEYK |
| | CKVSNKALPAPIEKTISKAKGQPRE |
| | PQVYTLPPSRDELTKNQVSLTCLVK |
| | GFYPSDIAVEWESNGQPENNYKTTP |
| | PVLDSDGSFFLYSKLTVDKSRWQQG |
| | NVFSCSVMHEALHNHYTQKSLSLSP |
| | KGGGGSGGGGSGGGGSGGGGSTIPP |
| | HVQKSVNNDMIVTDNNGAVKFPQLC |
| | KFCDVRESTCDNQKSCMSNCSITSI |
| | CEKPQEVCVAVWRKNDENITLETVC |
| | HDPKLPYHDFILEDAASPKCIMKEK |
| | KKPGETFFMCSCSSDECNDNIIFSE |
| | EYNTSNPD |
| | (SEQ ID NO: 74) |
| | DIQMTQSPSTLSASVGDRVTITCRA |
| | SQDISNYLNWYQQKPDQSPKLLIYY |
| | TSRLRSGVPARFSGSGSGTEYTLTI |
| | SSLQPDDFATYYCQQGNTLPPTFGQ |
| | GTKVEVKRTVAAPSVFIFPPSDEQL |
| | KSGTASVVCLLNNFYPREAKVQWKV |
| | DNALQSGNSQESVTEQDSKDSTYSL |
| | SSTLTLSKADYEKHKVYACEVTHQG |
| | LSSPVTKSENRGEC |
| | (SEQ ID NO: 65) |
| BJ-005-P3 | EVQLVQSGAEVKKPGSSVKVSCAAS |
| | GFNIKDNYIHWVRQAPGQGLEWIGW |
| | IDPANTNTKYDPKFQGKATITADTS |
| | TNTAYMELSSLRSEDTAVYYCARRG |
| | DGYRYFYAMAYWGQGTLVTVSSAST |
| | KGPSVFPLAPSSKSTSGGTAALGCL |
| | VKDYFPEPVTVSWNSGALTSGVHTF |
| | PAVLQSSGLYSLSSVVTVPSSSLGT |
| | QTYICNVNHKPSNTKVDKRVEPKSC |
| | DKTHTCPPCPAPELLGGPSVFLFPP |
| | KPKDTLMISRTPEVTCVVVDVSHED |
| | PEVKFNWYVDGVEVHNAKTKPREEQ |
| | YNSTYRVVSVLTVLHQDWLNGKEYK |
| | CKVSNKALPAPIEKTISKAKGQPRE |
| | PQVYTLPPSRDELTKNQVSLTCLVK |
| | GFYPSDIAVEWESNGQPENNYKTTP |
| | PVLDSDGSFFLYSKLTVDKSRWQQG |
| | NVFSCSVMHEALHNHYTQKSLSLSP |
| | KGGGGSGGGGSGGGGSGGGGSTIPP |
| | HVQKSVNNDMIVTDNNGAVKFPQLC |
| | KFCDVRESTCDNQKSCMSNCSITSI |
| | CEKPQEVCVAVWRKNDENITLETVC |
| | HDPKLPYHDFILEDAASPKCIMKEK |
| | KKPGETFFMCSCSSDECNDNIIFSE |
| | EYNTSNPD |
| | (SEQ ID NO: 75) |
| | DIQMTQSPSTLSASVGDRVTITCRA |
| | SQDISDYLNWYQQKPDQSPKLLIYH |
| | TSRLHSGVPARFSGSGSGTEYTLTI |
| | SSLQPDDFATYYCQQGNTLPPTFGQ |
| | GTKVEVKRTVAAPSVFIFPPSDEQL |
| | KSGTASVVCLLNNFYPREAKVQWKV |
| | DNALQSGNSQESVTEQDSKDSTYSL |
| | SSTLTLSKADYEKHKVYACEVTHQG |
| | LSSPVTKSFNRGEC |
| | (SEQ ID NO: 66) |
| BJ-005-P4 | QVQLQESGPGLVKPSETLSLTCTVT |
| | GDSITSGYWNWIRQPAGKGLEYMGY |
| | ISYTGSTYYNPSLKSRLTISRDTSK |
| | NQYSLKLSSVTAADTAVYYCGSQRE |
| | WLLHTDYWGQGTLVTVSSASTKGPS |
| | VFPLAPSSKSTSGGTAALGCLVKDY |
| | FPEPVTVSWNSGALTSGVHTFPAVL |
| | QSSGLYSLSSVVTVPSSSLGTQTYI |
| | CNVNHKPSNTKVDKRVEPKSCDKTH |
| | TCPPCPAPELLGGPSVFLFPPKPKD |
| | TLMISRTPEVTCVVVDVSHEDPEVK |
| | FNWYVDGVEVHNAKTKPREEQYNST |
| | YRVVSVLTVLHQDWLNGKEYKCKVS |
| | NKALPAPIEKTISKAKGQPREPQVY |
| | TLPPSRDELTKNQVSLTCLVKGFYP |
| | SDIAVEWESNGQPENNYKTTPPVLD |
| | SDGSFFLYSKLTVDKSRWQQGNVFS |
| | CSVMHEALHNHYTQKSLSLSPKGGG |
| | GSGGGGSGGGGSGGGGSTIPPHVQK |
| | SVNNDMIVTDNNGAVKFPQLCKFCD |
| | VRESTCDNQKSCMSNCSITSICEKP |
| | QEVCVAVWRKNDENITLETVCHDPK |
| | LPYHDFILEDAASPKCIMKEKKKPG |
| | ETFFMCSCSSDECNDNIIFSEEYNT |
| | SNPD |
| | (SEQ ID NO: 76) |
| | DIVMTQSPDSLAVSLGERATINCKS |
| | SQSLLYTSNQKNSLAWYQQKPGQPP |
| | KLLIYWASTRESGVPDRFSGSGSGT |
| | DFTLTISSLQAEDVAVYYCQQYYGY |
| | PYTFGQGTKLEIKRTVAAPSVFIFP |
| | PSDEQLKSGTASVVCLLNNFYPREA |
| | KVQWKVDNALQSGNSQESVTEQDSK |
| | DSTYSLSSTLTLSKADYEKHKVYAC |
| | EVTHQGLSSPVTKSFNRGEC |
| | (SEQ ID NO: 67) |
| BJ-005-P5 | EVQLVESGGGLVQPGGSLRLSCAAS |
| | GFNITDTYMHWVRQAPGKGLEWVGW |
| | IDPANGHTKSDPKFQGRFTISADTS |
| | TNTAYLQMNSLRAEDTAVYYCARRG |
| | DGYRYFYAMGYWGQGTLVTVSSAST |
| | KGPSVFPLAPSSKSTSGGTAALGCL |
| | VKDYFPEPVTVSWNSGALTSGVHTF |
| | PAVLQSSGLYSLSSVVTVPSSSLGT |
| | QTYICNVNHKPSNTKVDKRVEPKSC |
| | DKTHTCPPCPAPELLGGPSVFLFPP |
| | KPKDTLMISRTPEVTCVVVDVSHED |
| | PEVKFNWYVDGVEVHNAKTKPREEQ |
| | YNSTYRVVSVLTVLHQDWLNGKEYK |
| | CKVSNKALPAPIEKTISKAKGQPRE |
| | PQVYTLPPSRDELTKNQVSLTCLVK |
| | GFYPSDIAVEWESNGQPENNYKTTP |
| | PVLDSDGSFFLYSKLTVDKSRWQQG |
| | NVFSCSVMHEALHNHYTQKSLSLSP |
| | KGGGGSGGGGSGGGGSGGGGSTIPP |
| | HVQKSVNNDMIVTDNNGAVKFPQLC |
| | KFCDVRESTCDNQKSCMSNCSITSI |
| | CEKPQEVCVAVWRKNDENITLETVC |
| | HDPKLPYHDFILEDAASPKCIMKEK |
| | KKPGETFFMCSCSSDECNDNIIFSE |
| | EYNTSNPD |
| | (SEQ ID NO: 77) |
| | DIQMTQSPSSLSASVGDRVTITCRA |
| | SQDISNYLNWYQQKPGKAPELLIYY |
| | TSRLRSGVPSRFSGSGSGTDYTLTI |
| | SSLQPEDFATYYCQQGNTLPPTFGQ |
| | GTKVEIKRTVAAPSVFIFPPSDEQL |
| | KSGTASVVCLLNNFYPREAKVQWKV |
| | DNALQSGNSQESVTEQDSKDSTYSL |
| | SSTLTLSKADYEKHKVYACEVTHQG |
| | LSSPVTKSFNRGEC |
| | (SEQ ID NO: 68) |
| CP-ab2 | QVQLVQSGAEVKKPGASVKVSCKAS |
| | GFNITDTYMHWVRQAPGQRLEWMGW |
| | IDPANGHTKSDPKFQGRVTITADTS |
| | ASTAYMELSSLRSEDTAVYYCARRG |
| | DGYRYFYAMGYWGQGTTVTVSSAST |
| | KGPSVFPLAPSSKSTSGGTAALGCL |
| | VKDYFPEPVTVSWNSGALTSGVHTF |
| | PAVLQSSGLYSLSSVVTVPSSSLGT |
| | QTYICNVNHKPSNTKVDKRVEPKSC |
| | DKTHTCPPCPAPELLGGPSVFLFPP |
| | KPKDTLMISRTPEVTCVVVDVSHED |
| | PEVKFNWYVDGVEVHNAKTKPREEQ |
| | YNSTYRVVSVLTVLHQDWLNGKEYK |
| | CKVSNKALPAPIEKTISKAKGQPRE |
| | PQVYTLPPSRDELTKNQVSLTCLVK |
| | GFYPSDIAVEWESNGQPENNYKTTP |
| | PVLDSDGSFFLYSKLTVDKSRWQQG |
| | NVFSCSVMHEALHNHYTQKSLSLSP |
| | KGGGGSGGGGSGGGGSGGGGSTIPP |
| | HVQKSVNNDMIVTDNNGAVKFPQLC |
| | KFCDVRESTCDNQKSCMSNCSITSI |
| | CEKPQEVCVAVWRKNDENITLETVC |
| | HDPKLPYHDFILEDAASPKCIMKEK |
| | KKPGETFFMCSCSSDECNDNIIFSE |
| | EYNTSNPD |
| | (SEQ ID NO: 78) |
| | DIQMTQSPSSLSASVGDRVTITCRA |
| | SQDISNYLNWYQQKPGKAPKLLIYY |
| | TSRLRSGVPSRFSGSGSGTDYTFTI |
| | SSLQPEDIATYYCQQGNTLPPTFGQ |
| | GTKLEIKRTVAAPSVFIFPPSDEQL |
| | KSGTASVVCLLNNFYPREAKVQWKV |
| | DNALQSGNSQESVTEQDSKDSTYSL |
| | SSTLTLSKADYEKHKVYACEVTHQG |
| | LSSPVTKSFNRGEC |
| | (SEQ ID NO: 69) |

### Example 2: Analysis of Affinity to PD-L1 of Human and Cynomolgus Macaque

The exemplary sequences of the bifunctional molecule binding to human TGFβ and PD-L1 and monoclonal antibody or an antigen-binding fragment thereof binding to human PD-L1 involved in the present invention were as shown in the following Tables 2-6.

TABLE 2
CDR sequences of antibodies binding to human PD-L1 in bifunctional
molecules

| Name of | | | | | | |
|---|---|---|---|---|---|---|
| the | | | | | | |
| molecule it | | | | | | |
| belongs to | CDR1 | CDR2 | CDR3 | CDR1′ | CDR2′ | CDR3′ |
| BJ-005-P1/ | GFNITDTY | GWIDPANGH | RGDGYRY | RASQDIS | YTSRLRS | QQGNTL |
| BJ-005-P2/ | MH | TKSDPKFQG | FYAMGY | NYLN | (SEQ ID | PPT |
| BJ-005-P5/ | (SEQ ID | (SEQ ID NO: | (SEQ ID | (SEQ ID | NO: 13) | (SEQ ID |
| CP-ab2 | NO: 1) | 4) | NO: 7) | NO: 10) | | NO: 16) |
| BJ-005-P3 | GFNIKDN | GWIDPANTN | RGDGYRY | RASQDIS | HTSRLHS | QQGNTL |
| | YIH | TKYDPKFQG | FYAMAY | DYLN | (SEQ ID | PPT |
| | (SEQ ID | (SEQ ID NO: | (SEQ ID | (SEQ ID | NO: 14) | (SEQ ID |
| | NO: 2) | 5) | NO: 8) | NO: 11) | | NO: 16) |
| BJ-005-P4 | GDSITSGY | YISYTGSTY | GSQREWL | KSSQSLL | WASTRES | QQYYGY |
| | WN | YNPSLKS | LHTDY | YTSNQK | (SEQ ID | PYT |
| | (SEQ ID | (SEQ ID NO: | (SEQ ID | NSLA | NO: 15) | (SEQ ID |
| | NO: 3) | 6) | NO: 9) | (SEQ ID | | NO: 17) |
| | | | | NO: 12) | | |

TABLE 3
Framework region sequences of antibodies binding to human PD-L1 in
bifunctional molecules
Heavy chain framework region sequences

| Name of the | | | | |
|---|---|---|---|---|
| molecule it | | | | |
| belongs to | FR1 | FR2 | FR3 | FR4 |
| BJ-005-P1/ | EVQLVQSGAEV | WVRQAPG | KATITADTSTNTA | WGQGTLVTV |
| BJ-005-P2/ | KKPGSSVKVSCA | QGLEWI | YMELSSLRSEDTA | SS |
| BJ-005-P3 | AS | (SEQ ID NO: | VYYCAR | (SEQ ID NO: |
| | (SEQ ID NO: 18) | 22) | (SEQ ID NO: 26) | 30) |
| BJ-005-P4 | QVQLQESGPGLV | WIRQPAGK | RLTISRDTSKNQY | WGQGTLVTV |
| | KPSETLSLTCTV | GLEYMG | SLKLSSVTAADTA | SS |
| | T | (SEQ ID NO: | VYYC | (SEQ ID NO: |
| | (SEQ ID NO: 19) | 23) | (SEQ ID NO: 27) | 30) |
| BJ-005-P5 | EVQLVESGGGLV | WVRQAPG | RFTISADTSTNTA | WGQGTLVTV |
| | QPGGSLRLSCAA | KGLEWV | YLQMNSLRAEDT | SS |
| | S | (SEQ ID NO: | AVYYCAR | (SEQ ID NO: |
| | (SEQ ID NO: 20) | 24) | (SEQ ID NO: 28) | 30) |
| | QVQLVQSGAEV | WVRQAPG | RVTITADTSASTA | WGQGTTVTV |
| | KKPGASVKVSC | QRLEWM | YMELSSLRSEDTA | SS |
| | KAS | (SEQ ID NO: | VYYCAR | (SEQ ID NO: |
| | (SEQ ID NO: 21) | 25) | (SEQ ID NO: 29) | 31) |

Light chain framework region sequences

| Name of the | | | | |
|---|---|---|---|---|
| molecule it | | | | |
| belongs to | FR1′ | FR2′ | FR3′ | FR4′ |
| BJ-005-P1 | DIQMTQSPSTLS | WYQQKPG | GVPARFSGSGSGT | FGQGTKVEV |
| | ASVGDRVTITC | KAPKLLIY | EYTLTISSLQPDDF | K |
| | (SEQ ID NO: 32) | (SEQ ID NO: | ATYYC | (SEQ ID NO: |
| | | 35) | (SEQ ID NO: 39) | 43) |
| BJ-005-P2/B | DIQMTQSPSTLS | WYQQKPD | GVPARFSGSGSGT | FGQGTKVEV |
| J-005-P3 | ASVGDRVTITC | QSPKLLIY | EYTLTISSLQPDDF | K |
| | (SEQ ID NO: 32) | (SEQ ID NO: | ATYYC | (SEQ ID NO: |
| | | 36) | (SEQ ID NO: 39) | 43) |
| BJ-005-P4 | DIVMTQSPDSLA | WYQQKPG | GVPDRFSGSGSGT | FGQGTKLEIK |
| | VSLGERATINC | QPPKLLIY | DFTLTISSLQAEDV | (SEQ ID NO: |
| | (SEQ ID NO: 33) | (SEQ ID NO: | AVYYC | 44) |
| | | 37) | (SEQ ID NO: 40) | |
| BJ-005-P5 | DIQMTQSPSSLS | WYQQKPG | GVPSRFSGSGSGT | FGQGTKVEIK |
| | ASVGDRVTITC | KAPELLIY | DYTLTISSLQPEDF | (SEQ ID NO: |
| | (SEQ ID NO: 34) | (SEQ ID NO: | ATYYC | 45) |
| | | 38) | (SEQ ID NO: 41) | |
| CP-ab2 | DIQMTQSPSSLS | WYQQKPG | GVPSRFSGSGSGT | FGQGTKLEIK |
| | ASVGDRVTITC | KAPKLLIY | DYTFTISSLQPEDI | (SEQ ID NO: |
| | (SEQ ID NO: 34) | (SEQ ID NO: | ATYYC | 44) |
| | | 35) | (SEQ ID NO: 42) | |

TABLE 4
Variable region sequences of antibodies binding to human PD-L1 in
bifunctional molecules

| Name of the | Heavy chain variable region sequences |
|---|---|
| molecule it | (wherein CDR sequences are |
| belongs to | underlined) |
| BJ-005-P1/ | EVQLVQSGAEVKKPGSSVKVSCAASGFNITDTYMHWVRQAPGQGLE |
| BJ-005-P2 | WIGWIDPANGHTKSDPKFQGKATITADTSTNTAYMELSSLRSEDTA |
| | VYYCARRGDGYRYFYAMGYWGQGTLVTVSS (SEQ ID NO: 46) |
| BJ-005-P3 | EVQLVQSGAEVKKPGSSVKVSCAASGFNIKDNYIHWVRQAPGQGLEW |
| | IGWIDPANTNTKYDPKFQGKATITADTSTNTAYMELSSLRSEDTAVY |
| | YCARRGDGYRYFYAMAYWGQGTLVTVSS (SEQ ID NO: 47) |
| BJ-005-P4 | QVQLQESGPGLVKPSETLSLTCTVTGDSITSGYWNWIRQPAGKGLEYM |
| | GYISYTGSTYYNPSLKSRLTISRDTSKNQYSLKLSSVTAADTAVYYCG |
| | SQREWLLHTDYWGQGTLVTVSS (SEQ ID NO: 48) |
| BJ-005-P5 | EVQLVESGGGLVQPGGSLRLSCAASGFNITDTYMHWVRQAPGKGLEW |
| | VGWIDPANGHTKSDPKFQGRFTISADTSTNTAYLQMNSLRAEDTAVY |
| | YCARRGDGYRYFYAMGYWGQGTLVTVSS (SEQ ID NO: 49) |
| CP-ab2 | QVQLVQSGAEVKKPGASVKVSCKASGFNITDTYMHWVRQAPGQRLEW |
| | MGWIDPANGHTKSDPKFQGRVTITADTSASTAYMELSSLRSEDTAVY |
| | YCARRGDGYRYFYAMGYWGQGTTVTVSS (SEQ ID NO: 50) |
| Name of the | Light chain variable region sequences |
| molecule it | (wherein CDR sequences are |
| belongs to | underlined) |
| BJ-005-P1 | DIQMTQSPSTLSASVGDRVTITCRASQDISNYLNWYQQKPGKAPKLLI |
| | YYTSRLRSGVPARFSGSGSGTEYTLTISSLQPDDFATYYCQQGNTLPP |
| | TFGQGTKVEVK (SEQ ID NO: 51) |
| BJ-005-P2 | DIQMTQSPSTLSASVGDRVTITCRASQDISNYLNWYQQKPDQSPKLLIY |
| | YTSRLRSGVPARFSGSGSGTEYTLTISSLQPDDFATYYCQQGNTLPPTF |
| | GQGTKVEVK (SEQ ID NO: 52) |
| BJ-005-P3 | DIQMTQSPSTLSASVGDRVTITCRASQDISDYLNWYQQKPDQSPKLLIY |
| | HTSRLHSGVPARFSGSGSGTEYTLTISSLQPDDFATYYCQQGNTLPPTF |
| | GQGTKVEVK (SEQ ID NO: 53) |
| BJ-005-P4 | DIVMTQSPDSLAVSLGERATINCKSSQSLLYTSNQKNSLAWYQQKPGQ |
| | PPKLLIYWASTRESGVPDRFSGSGSGTDFTLTISSLQAEDVAVYYCQQ |
| | YYGYPYTFGQGTKLEIK (SEQ ID NO: 54) |
| BJ-005-P5 | DIQMTQSPSSLSASVGDRVTITCRASQDISNYLNWYQQKPGKAPELLIY |
| | YTSRLRSGVPSRFSGSGSGTDYTLTISSLQPEDFATYYCQQGNTLPPTF |
| | GQGTKVEIK (SEQ ID NO: 55) |
| CP-ab2 | DIQMTQSPSSLSASVGDRVTITCRASQDISNYLNWYQQKPGKAPKLLIY |
| | YTSRLRSGVPSRFSGSGSGTDYTFTISSLQPEDIATYYCQQGNTLPPTF |
| | GQGTKLEIK (SEQ ID NO: 56) |

TABLE 5
Constant region sequences of
antibodies binding to human PD-L1 in
bifunctional molecules

| Heavy chain | Light chain |
|---|---|
| constant | constant region |
| region sequence | sequence |
| ASTKGPSVFPLAPSSKSTSG | RTVAAPSVFIFPPSDEQLKS |
| GTAALGCLVKDYFPEPVTVS | GTASVVCLLNNFYPREAKVQ |
| WNSGALTSGVHTFPAVLQSS | WKVDNALQSGNSQESVTEQD |
| GLYSLSSVVTVPSSSLGTQT | SKDSTYSLSSTLTLSKADYE |
| YICNVNHKPSNTKVDKRVEP | KHKVYACEVTHQGLSSPVTK |
| KSCDKTHTCPPCPAPELLGG | SENRGEC |
| PSVFLFPPKPKDTLMISRTP | (SEQ ID NO: 58) |
| EVTCVVVDVSHEDPEVKFNW | |
| YVDGVEVHNAKTKPREEQYN | |
| STYRVVSVLTVLHQDWLNGK | |
| EYKCKVSNKALPAPIEKTIS | |
| KAKGQPREPQVYTLPPSRDE | |
| LTKNQVSLTCLVKGFYPSDI | |
| AVEWESNGQPENNYKTTPPV | |
| LDSDGSFFLYSKLTVDKSRW | |
| QQGNVFSCSVMHEALHNHYT | |
| QKSLSLSPK | |
| (SEQ ID NO: 57) | |

TABLE 6
Sequences of antibodies binding to human
PD-L1 in bifunctional molecules

| | Heavy chain sequences |
|---|---|
| Name of the | (wherein CDR sequences are |
| molecule it | underlined and constant |
| belongs to | region sequences are italicized) |
| BJ-005-P1/ | EVQLVQSGAEVKKPGSSVKVSCAASGFNITDTY |
| BJ-005-P2 | MHWVRQAPGQGLEWIGWIDPANGHTKSDPKFQG |
| | KATITADTSTNTAYMELSSLRSEDTAVYYCARR |
| | GDGYRYFYAMGYWGQGTLVTVSSASTKGPSVFP |
| | LAPSSKSTSGGTALALGCLVKDYFPEPVTVSWN |
| | SGALTSGVHTFPAVLQSSGLYSLSSVVTVPSSS |
| | GTQTYICNVNHKPSNTKVDKRVEPKSCDKTHTC |
| | PPCPAPELLGGPSVFLFPPKPKDTLMISRTPEV |
| | TCVVVDVSHEDPEVKFNWYVDGVEVHNAKTKPR |
| | EEQYNSTYRVVSVLTVLHQDWLNGKEYKCKVSN |
| | KALPAPIEKTISKAKGQPREPQVYTLPPSRDEL |
| | TKNQVSLTCLVKGFYPSDIAVEWESNGQPENNY |
| | KTTPPVLDSDGSFFLYSKLTVDKSRWQQGNVFS |
| | CSVMHEALHNHYTQKSLSLSPK |
| | (SEQ ID NO: 59) |
| BJ-005-P3 | EVQLVQSGAEVKKPGSSVKVSCAASGFNIKDNY |
| | IHWVRQAPGQGLEWIGWIDPANTNTKYDPKFQG |
| | KATITADTSTNTAYMELSSLRSEDTAVYYCARR |
| | GDGYRYFYAMAYWGQGTLVTVSSASTKGPSVFP |
| | LAPSSKSTSGGTAALGCLVKDYFPEPVTVSWNS |
| | GALTSGVHTFPAVLQSSGLYSLSSVVTVPSSSL |
| | GTQTYICNVNHKPSNTKVDKRVEPKSCDKTHTC |
| | PPCPAPELLGGPSVFLFPPKPKDTLMISRTPEV |
| | TCVVVDVSHEDPEVKFNWYVDGVEVHNAKTKPR |
| | EEQYNSTYRVVSVLTVLHQDWLNGKEYKCKVSN |
| | KALPAPIEKTISKAKGQPREPQVYTLPPSRDEL |
| | TKNQVSLTCLVKGFYPSDIAVEWESNGQPENNY |
| | KTTPPVLDSDGSFFLYSKLTVDKSRWQQGNVFS |
| | CSVMHEALHNHYTQKSLSLSPK |
| | (SEQ ID NO: 60) |
| BJ-005-P4 | QVQLQESGPGLVKPSETLSLTCTVTGDSITSGY |
| | WNWIRQPAGKGLEYMGYISYTGSTYYNPSLKSR |
| | LTISRDTSKNQYSLKLSSVTAADTAVYYCGSQR |
| | EWLLHTDYWGQGTLVTVSSASTKGPSVFPLAPS |
| | SKSTSGGTAALGCLVKDYFPEPVTVSWNSGALT |
| | SGVHTFPAVLQSSGLYSLSSVVTVPSSSLGTQT |
| | YICNVNHKPSNTKVDKRVEPKSCDKTHTCPPCP |
| | APELLGGPSVFLFPPKPKDTLMISRTPEVTCVV |
| | VDVSHEDPEVKFNWYVDGVEVHNAKTKPREEQY |
| | NSTYRVVSVLTVLHQDWLNGKEYKCKVSNKALP |
| | APIEKTISKAKGQPREPQVYTLPPSRDELTKNQ |
| | VSLTCLVKGFYPSDIAVEWESNGQPENNYKTTP |
| | PVLDSDGSFFLYSKLTVDKSRWQQGNVFSCSVM |
| | HEALHNHYTQKSLSLSPK |
| | (SEQ ID NO: 61) |
| BJ-005-P5 | EVQLVESGGGLVQPGGSLRLSCAASGFNITDTY |
| | MHWVRQAPGKGLEWVGWIDPANGHTKSDPKFQG |
| | RFTISADTSTNTAYLQMNSLRAEDTAVYYCARR |
| | GDGYRYFYAMGYWGQGTLVTVSSASTKGPSVFP |
| | LAPSSKSTSGGTAALGCLVKDYFPEPVTVSWNS |
| | GALTSGVHTFPAVLQSSGLYSLSSVVTVPSSSL |
| | GTQTYICNVNHKPSNTKVDKRVEPKSCDKTHTC |
| | PPCPAPELLGGPSVFLFPPKPKDTLMISRTPEV |
| | TCVVVDVSHEDPEVKFNWYVDGVEVHNAKTKPR |
| | EEQYNSTYRVVSVLTVLHQDWLNGKEYKCKVSN |
| | KALPAPIEKTISKAKGQPREPQVYTLPPSRDEL |
| | TKNQVSLTCLVKGFYPSDIAVEWESNGQPENNY |
| | KTTPPVLDSDGSFFLYSKLTVDKSRWQQGNVFS |
| | CSVMHEALHNHYTQKSLSLSPK |
| | (SEQ ID NO: 62) |
| CP-ab2 | QVQLVQSGAEVKKPGASVKVSCKASGFNITDTY |
| | MHWVRQAPGQRLEWMGWIDPANGHTKSDPKFQG |
| | RVTITADTSASTAYMELSSLRSEDTAVYYCARR |
| | GDGYRYFYAMGYWGQGTTVTVSSASTKGPSVFP |
| | LAPSSKSTSGGTAALGCLVKDYFPEPVTVSWNS |
| | GALTSGVHTFPAVLQSSGLYSLSSVVTVPSSSL |
| | GTQTYICNVNHKPSNTKVDKRVEPKSCDKTHTC |
| | PPCPAPELLGGPSVFLFPPKPKDTLMISRTPEV |
| | TCVVVDVSHEDPEVKFNWYVDGVEVHNAKTKPR |
| | EEQYNSTYRVVSVLTVLHQDWLNGKEYKCKVSN |
| | KALPAPIEKTISKAKGQPREPQVYTLPPSRDEL |
| | TKNQVSLTCLVKGFYPSDIAVEWESNGQPENNY |
| | KTTPPVLDSDGSFFLYSKLTVDKSRWQQGNVFS |
| | CSVMHEALHNHYTQKSLSLSPK |
| | (SEQ ID NO: 63) |
| Name of the | Light chain sequences |
| molecule it | (wherein CDR sequences |
| belongs to | are underlined and constant |
| | region sequences are italicized) |
| BJ-005-P1 | DIQMTQSPSTLSASVGDRVTITCRASQDISNYL |
| | NWYQQKPGKAPKLLIYYTSRLRSGVPARFSGSG |
| | SGTEYTLTISSLQPDDFATYYCQQGNTLPPTFG |
| | QGTKVEVKRTVAAPSVFIFPPSDEQLKSGTASV |
| | VCLLNNFYPREAKVQWKVDNALQSGNSQESVTE |
| | QDSKDSTYSLSSTLTLSKADYEKHKVYACEVTH |
| | QGLSSPVTKSFNRGEC |
| | (SEQ ID NO: 64) |
| BJ-005-P2 | DIQMTQSPSTLSASVGDRVTITCRASQDISNYL |
| | NWYQQKPDQSPKLLIYYTSRLRSGVPARFSGSG |
| | SGTEYTLTISSLQPDDFATYYCQQGNTLPPTFG |
| | QGTKVEVKRTVAAPSVFIFPPSDEQLKSGTASV |
| | VCLLNNFYPREAKVQWKVDNALQSGNSQESVTE |
| | QDSKDSTYSLSSTLTLSKADYEKHKVYACEVTH |
| | QGLSSPVTKSFNRGEC |
| | (SEQ ID NO: 65) |
| BJ-005-P3 | DIQMTQSPSTLSASVGDRVTITCRASQDISDYL |
| | NWYQQKPDQSPKLLIYHTSRLHSGVPARFSGSG |
| | SGTEYTLTISSLQPDDFATYYCQQGNTLPPTFG |
| | QGTKVEVKRTVAAPSVFIFPPSDEQLKSGTASV |
| | VCLLNNFYPREAKVQWKVDNALQSGNSQESVTE |
| | QDSKDSTYSLSSTLTLSKADYEKHKVYACEVTH |
| | QGLSSPVTKSFNRGEC |
| | (SEQ ID NO: 66) |
| BJ-005-P4 | DIVMTQSPDSLAVSLGERATINCKSSQSLLYTS |
| | NQKNSLAWYQQKPGQPPKLLIYWASTRESGVPD |
| | RFSGSGSGTDFTLTISSLQAEDVAVYYCQQYYG |
| | YPYTFGQGTKLEIKRTVAAPSVFIFPPSDEQLK |
| | SGTASVVCLLNNFYPREAKVQWKVDNALQSGNS |
| | QESVTEQDSKDSTYSLSSTLTLSKADYEKHKVY |
| | ACEVTHQGLSSPVTKSFNRGEC |
| | (SEQ ID NO: 67) |
| BJ-005-P5 | DIQMTQSPSSLSASVGDRVTITCRASQDISNYL |
| | NWYQQKPGKAPELLIYYTSRLRSGVPSRFSGSG |
| | SGTDYTLTISSLQPEDFATYYCQQGNTLPPTFG |
| | QGTKVEIKRTVAAPSVFIFPPSDEQLKSGTASV |
| | VCLLNNFYPREAKVQWKVDNALQSGNSQESVTE |
| | QDSKDSTYSLSSTLTLSKADYEKHKVYACEVTH |
| | QGLSSPVTKSFNRGEC |
| | (SEQ ID NO: 68) |
| CP-ab2 | DIQMTQSPSSLSASVGDRVTITCRASQDISNYL |
| | NWYQQKPGKAPKLLIYYTSRLRSGVPSRFSGSG |
| | SGTDYTFTISSLQPEDIATYYCQQGNTLPPTFG |
| | QGTKLEIKRTVAAPSVFIFPPSDEQLKSGTASV |
| | VCLLNNFYPREAKVQWKVDNALQSGNSQESVTE |
| | QDSKDSTYSLSSTLTLSKADYEKHKVYACEVTH |
| | QGLSSPVTKSFNRGEC |
| | (SEQ ID NO: 69) |

The full sequence of control BJ-007 molecule (equivalent to M7824 Bintrafusp alfa) is as shown below, wherein the sequence of the heavy chain part is underlined and TGFbetaRII is italicized:

| | |
|---|---|
| Heavy chain + linker + | EVQLLESGGGLVOPGGSLRLSCAAS |
| TGFbetaRII | GFTESSYIMMWVRQAPGKGLEWVSS |
| (SEQ ID NO: 72) | IYPSGGITFYADTVKGRFTISRDNS |
| | KNTLYLOMNSLRAEDTAVYYCARIK |
| | LGTVTTVDYWGQGTLVTVSSASTKG |
| | PSVFPLAPSSKSTSGGTAALGCLVK |
| | DYFPEPVTVSWNSGALTSGVHTFPA |
| | VLOSSGLYSLSSVVTVPSSSLGTQT |
| | YICNVNHKPSNTKVDKKVEPKSCDK |
| | THTCPPCPAPELLGGPSVFLFPPKP |
| | KDTLMISRTPEVTCVVVDVSHEDPE |
| | VKFNWYVDGVEVHNAKTKPREEQYN |
| | STYRVVSVLTVLHQDWLNGKEYKCK |
| | VSNKALPAPIEKTISKAKGQPREPQ |
| | VYTLPPSRDELTKNQVSLTCLVKGF |
| | YPSDIAVEWESNGQPENNYKTTPPV |
| | LDSDGSFFLYSKLTVDKSRWQQGNV |
| | ESCSVMHEALHNHYTQKSLSLSPGG |
| | sggggsggggsggggTIPPHVQKSV |
| | NNDMIVTDNNGAVKFPQLCKFCDVR |
| | FSTCDNQKSCMSNCSITSICEKPQE |
| | VCVAVWRKNDENITLETVCHDPKLP |
| | YHDFILEDAASPKCIMKEKKKPGET |
| | FFMCSCSSDECNDNIIFSEEYNTSN |
| | PD |
| Light chain | QSALTQPASVSGSPGQSITISCTGT |
| (SEQ ID NO: 73) | SSDVGGYNYVSWYQQHPGKAPKLMI |
| | YDVSNRPSGVSNRFSGSKSGNTASL |
| | TISGLQAEDEADYYCSSYTSSSTRV |
| | FGTGTKVTVLGQPKANPTVTLFPPS |
| | SEELQANKATLVCLISDFYPGAVTV |
| | AWKADGSPVKAGVETTKPSKQSNNK |
| | YAASSYLSLTPEQWKSHRSYSCQVT |
| | HEGSTVEKTVAPTECS |

The affinity of different bifunctional molecules to PD-L1 of human and Cynomolgus Macaque was measured with a Gator instrument (https://probelife.com.gator.html), and the results are as shown in the following table.

TABLE 7
Affinity of Different Molecules
to Human and Cynomolgus Macaque

| | Human PD-L1 | Cynomolgus Macaque PD-L1 |
|---|---|---|
| Molecule | Kd | Kd |
| BJ-005-P1 | 8.03 × 10⁻¹⁰ | 8.03 × 10⁻¹⁰ |
| BJ-005-P2 | 1.26 × 10⁻¹⁰ | 1.55 × 10⁻¹⁰ |
| BJ-005-P3 | 4.62 × 10⁻¹¹ | 2.02 × 10⁻¹⁰ |
| BJ-005-P4 | 1.75 × 10⁻¹⁰ | 3.08 × 10⁻¹⁰ |
| BJ-005-P5 | 2.05 × 10⁻¹⁰ | 4.09 × 10⁻¹⁰ |
| BJ-007 | 5.96 × 10⁻¹⁰ | 7.33 × 10⁻¹⁰ |

It can be seen from Table 7 that the equilibrium dissociation constant of BJ-005-P1 was equivalent to that of BJ-007, while the equilibrium dissociation constants of BJ-005-P2, BJ-005-P3, BJ-005-P4 and BJ-005-P5 were all significantly smaller than that of BJ-007, indicating that BJ-005-P2, BJ-005-P3, BJ-005-P4 and BJ-005-P5 had higher binding affinity.

### Example 3: Mixed Lymphocyte Reaction (MLR)

The effects of different molecules on T cell activation were tested by MLR.

Method: Fresh PBMC was purchased from Allcells and used for inducing monocyte-derived dendritic cells (mo-DC). CD3⁺ T cells were isolated from fresh PBMC of another donor. The CD3⁺ T (responder) cells and the mo-DC (stimulator) were distributed into a 96-well plate, and then ten-fold serial dilutions of four molecules (with final concentrations of 0.0001, 0.001, 0.01, 1 and 10 μg/mL) or Atezolizumab (with a final concentration of 10 μg/mL, as a positive control) were added into specified wells, and the 96-well plate was co-cultured at 37° C. and 5% CO₂. The supernatant was collected, and the concentration of IL-2 after 3 days of culture and the concentration of IFN-γ after 5 days of culture were detected by ELISA, respectively.

Specific experimental steps: fresh PBMC was purchased from Allcells; the PBMC was inoculated into a 100 mm cell culture dish (10 mL/dish) with a 1640 medium (serum-free) at a density of 2.5E6/mL, and incubated in a 5% CO₂ incubator at 37° C. for 2 hours to collect monocytes; then the supernatant was discarded, and then the cells were washed twice with DPBS; and 10 mL of a 1640 complete medium containing 50 ng/mL of IL-4 and 100 ng/mL of GM-CSF was added into the cell culture dish, and then the cells were incubated in a 5% CO₂ incubator at 37° C. (day 0). On day 3, half of the cell culture medium was replaced by a fresh 1640 complete medium containing IL-4 and GM-CSF. Immature mo-DC was treated with 1 μg/mL of LPS on day 6. On day 7, mature mo-DC was collected from the cell culture dish, and treated with 10 μg/mL of mitomycin C at a density of 1E6/mL at 37° C. for 1.5 hours, and then the mo-DC was washed with 1640 for 3 times. Then it was resuspended in a 1640 complete medium. CD3+ T cells were isolated from another donor and resuspended in a 1640 medium. DC (5E4/well/50 μL) and CD3+ T cells (2E5/well/100 μL) were distributed into a 96-well plate. 50 μL of serial dilution molecules (with final concentrations of 0.0001, 0.001, 0.01, 0.1, 1 and 10 μg/mL) or 50 μL of Atezoluzumab (with a final concentration of 10 μg/mL) were added into the well plate. The supernatant was collected for detection of IL-2 after 3 days of incubation, and for detection of IFN-γ after 5 days of incubation, by ELISA.

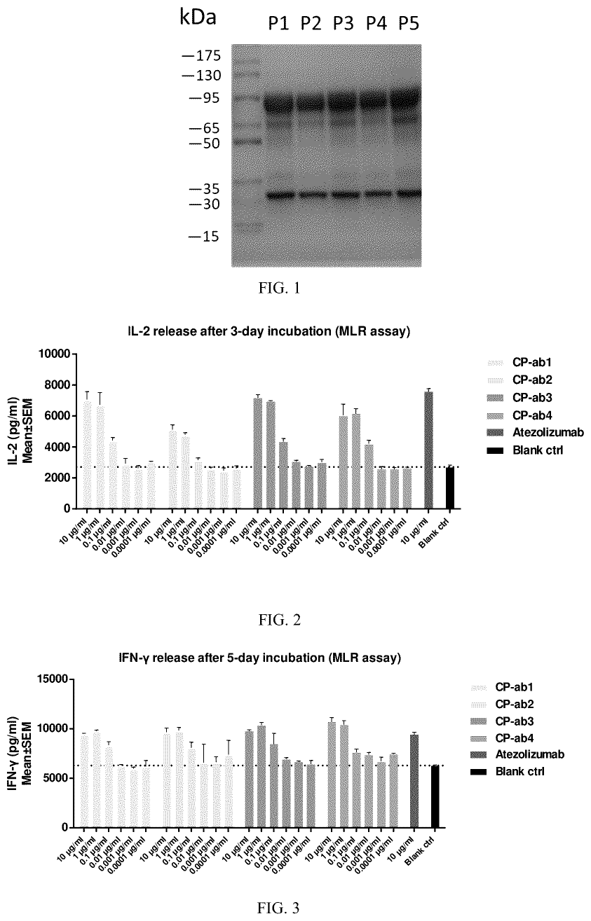

Results: As shown in FIGS. 2-3, Atezolizumab increased the release of IL-2 and IFN-γ as expected. Compared with the control group, CP-ab1 (BJ-007), CP-ab2 (14H2/14L2), CP-ab3 (BJ-005-P2) and CP-ab4 (BJ-005-P5) at concentrations of 0.1, 1 and 10 μg/mL respectively all promoted the release of IL-2 and IL-5.

### Example 4: TGFβ Blocking Experiment

Cell: HEK-Blue™ TGF-β cells, purchased from InvivoGen.

Experimental process: On the first day, into each well of a well plate added was 150 ul of a cell suspension (about 50,000 cells); different molecules with a starting concentration of 58 nM were diluted by 3 times in duplicate with a concentration gradient of 10; then each well was added with 50 ul of the molecule, and added with 0.04 nm of TGF-β1; the total volume of each well was 200 ul; and the plate was incubated in a CO₂ incubator at 37° C. for 20-24 hours. On the next day, each well was added with 160 μl of a QUANTI-Blue™ solution and 40 μl of a SEAP expression cell supernatant. The well was incubated at 37° C. for 1 hour. SEAP level was measured at 620 nm.

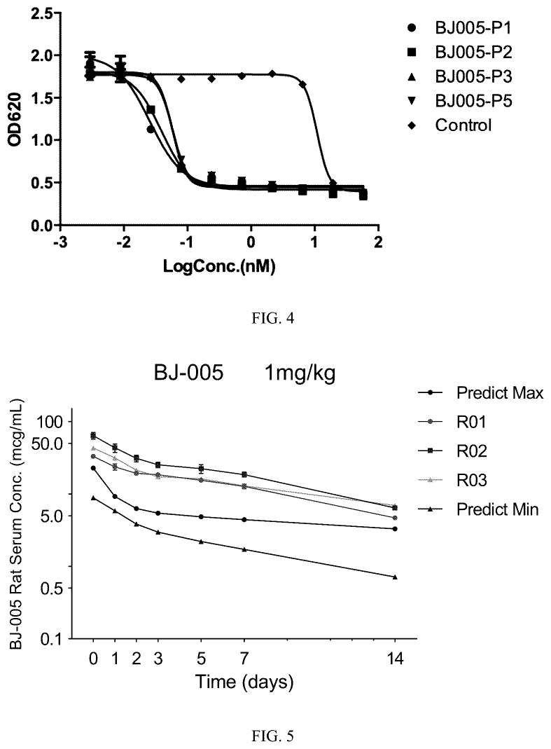

Results: as can be seen from Table 8 below and FIG. 4, IC₅₀s of BJ-005-P1, BJ-005-P2, BJ-005-P3 and BJ-005-P4 were significantly lower than that of the control group, indicating that the specificity and sensitivity of BJ-005-P1, BJ-005-P2, BJ-005-P3 and BJ-005-P4 were significantly stronger than those of the control group.

TABLE 8
IC₅₀s of different molecules

| Molecule | IC₅₀ (nM) |
|---|---|
| BJ-005-P1 | 0.0256 |
| BJ-005-P2 | 0.0388 |
| BJ-005-P3 | 0.0579 |
| BJ-005-P4 | 0.0587 |
| Control (BJ-007) | >10 nM |

### Example 5: Pharmacokinetic Analysis

Experimental animals: Male SD rats, 240 g-260 g.

Experimental equipments: a Microplate Reader 51119080 from Thermo Fisher; a Shaker TS-2 from Kylin-Bell; a Microplate dehydrator BIOS-401 from Bioscience; and an Automatic washer Elx405 Select CW from BioTek.

Reagents/materials: a goat anti-human TGFβRII from R&D; a goat anti-human Fc cross absorbed HRP conjugated label from Invitrogen; TMB from Solarbio; a termination solution of 2M H2SO4; PBS (pH 7.4); and PBS-0.05% Tween20 (PBST).

Experimental Method:

(1) Animal operation: Rats were weighed and numbered as R01 to R12. According to body weights, BJ-005-P5 or BJ-007 (1 mg/kg or 5 mg/kg) was injected into the rats through tail vein injection, wherein the rats numbered R01, R02 and R03 were injected with BJ-005-P5 at 1 mg/kg, rats numbered R04, R05 and R06 were injected with BJ-005-P5 at 5 mg/kg, rats numbered R07, R08 and R09 were injected with BJ-007 at 1 mg/kg, and rats numbered R010, R11 and R12 were injected with BJ-007 at 5 mg/kg. Blood was taken from jugular vein according to the following time points: 4, 24, 48, 72, 120, 168, and 336 hours. The blood was centrifuged at 3000×g and 4° C. for 10 min, and then the plasma was separated and stored at −80° C. After the plasma was prepared at all time points, detection by ELISA was conducted.

(2) Sample dilution: the sample was diluted according to the table below. Standard curve: starting from 125 ng/mL, it was diluted twice with a plasma/PBS buffer.

| | |
|---|---|
| Blood | Dilution multiple |

| | | |
|---|---|---|
| collection time | 5 mg/kg | 1 mg/kg |

| | | | |
|---|---|---|---|
| 4 | h | 5,000 | 800 |
| 24 | h | 2,000 | 400 |
| 48 | h | 2,000 | 400 |
| 72 | h | 2,000 | 400 |
| 120 | h | 1,000 | 200 |
| 168 | h | 1,000 | 200 |
| 336 | h | 1,000 | 200 |

(3) Detection by ELISA: The goat anti-human TGFβRII was diluted to 0.5 μg/mL with PBS. Into a 96 well plate added was 100 μL of the diluted antibody solution, and incubated overnight at 4° C. in the dark. The antigen solution was discarded. The wells were washed with 300 μL of PBST repeatedly for 3 times. Into each well of the 96 well plate added was 100 μL of a blocking buffer (PBST+1% BSA, w/v). The plate was shaken on a plate oscillator at 25° C.±3° C. and 220±20 rpm for 1 hour±20 minutes. The blocking buffer (PBST+1% BSA, w/v) was discarded. The wells were washed with 300 μL of PBST repeatedly for 3 times. For each well, 100 μL of diluted plasma was added. The plate was shaken on a plate oscillator at 25° C.±3° C. and 220±20 rpm for 1 hour±20 minutes. The plasma sample was discarded. The wells were washed with 300 μL of PBST repeatedly for 3 times. Into each well added was 100 μL of a second antibody (1:2,000 dilution of goat anti-human Fc cross absorbed HRP). The plate was shaken on a plate oscillator at 25° C.±3° C. and 220±20 rpm for 1 hour±20 minutes. The secondary antibody was labeled. The wells were washed with 300 μL of PBST repeatedly for 3 times. Into each well added was 100 μL of a freshly prepared TMB substrate. The wells were incubated at 25° C.±3° C. for 5-20 minutes. Into each well added was 50 μL of 2N H₂SO₄ to terminate the reaction. The plate was read at OD₄₅₀ nm.

(4) Plotting of concentration-time curve: In a coordinate graph with time as the horizontal coordinate and blood concentration as the vertical coordinate, the determined and calculated concentration values were taken as points and connected to form lines, so as to obtain concentration-time curves, as shown in FIGS. 5 to 8, wherein predict max is the predicted maximum value and predict min is the predicted minimum value. On the curve, the time corresponding to the half decrease of the blood drug concentration was an average drug clearance half-life of the drug (half life, t_{1/2}).

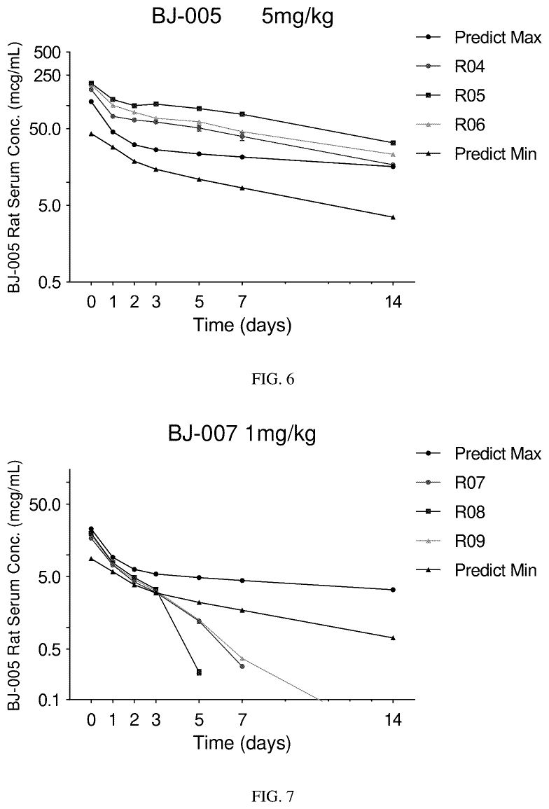

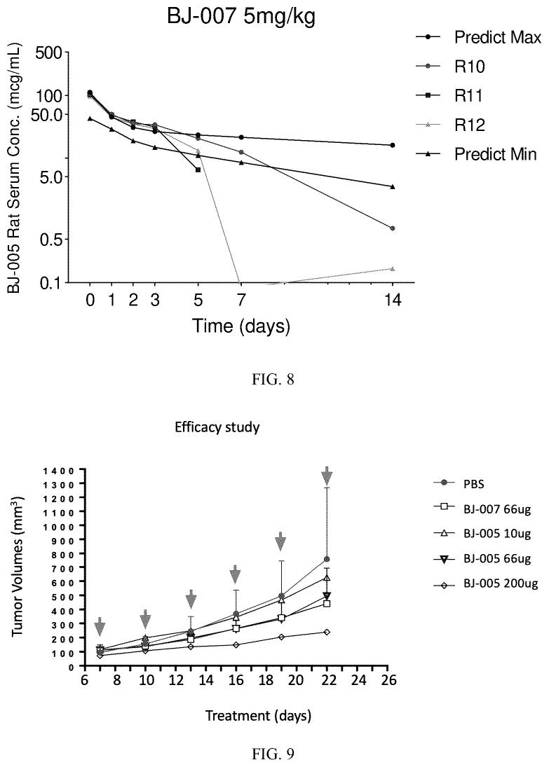

Results: as can be seen from FIGS. 5-8, at a dose level of 1 mg/kg, the average drug clearance half-life of BJ-005-P5 was 5.8 days (FIG. 5), while the average drug clearance half-life of BJ-007 was 1.2 days (FIG. 7); at a dose level of 5 mg/kg, the average drug clearance half-life of BJ-005-P5 was 6.1 days (FIG. 6), while the average drug clearance half-life of BJ-007 was 1.7 days (FIG. 8), indicating that the time required for the blood concentration of BJ-005-P5 to decrease by half was significantly longer than that of BJ-007.

### Example 6: Effect of BJ-005-P5 on Tumor Growth

Experimental animals: mice with double knock-in of human PD-L1 and PD-1, 6 weeks old and weighed about 20 g.

Experimental process: On day 0, 1 million MC38-hPD-L1 cells were transplanted into each mouse. When the tumor reached about 50-100 mm³, the following treatments were conducted (n=6): PBS; 10 ug of BJ-005-P5 every three days; 66 ug of BJ-005-P5 every three days; 200 ug of BJ-005-P5 every three days; 66 ug of BJ-007 every three days. The tumor volume and weight were measured every 3 days.

Results: it can be seen from FIG. 9 that both BJ-005-P5 and BJ-007 could inhibit the growth of tumor, and the inhibition effect was enhanced in a dose-dependent manner.

It should be understood that although the present invention has been specifically disclosed through preferred embodiments and optional features, modifications, improvements and changes can be made to the present invention disclosed herein by those skilled in the art, and these modifications, improvements and changes are considered to be within the scope of the present invention. The materials, methods and examples provided herein are representative of the preferred embodiments, are exemplary, and are not intended as limitations on the scope of the present technology.

1. A bifunctional molecule binding to human TGFβ and human PD-L1, comprising
(a) a human transforming growth factor β receptor II (TGFβRII) or a functional fragment thereof capable of binding to human TGFβ, and
(b) an antibody or an antigen-binding fragment thereof binding to human programmed death-ligand 1 (PD-L1),
wherein the antibody or the antigen-binding fragment thereof comprises:
a heavy chain complementarity determining region CDR1 comprising an amino acid sequence selected from SEQ ID NO: 1, SEQ ID NO: 2 and SEQ ID NO: 3 or an equivalent variant thereof;
a heavy chain complementarity determining region CDR2 comprising an amino acid sequence selected from SEQ ID NO: 4, SEQ ID NO: 5 and SEQ ID NO: 6 or an equivalent variant thereof;
a heavy chain complementarity determining region CDR3 comprising an amino acid sequence selected from SEQ ID NO: 7, SEQ ID NO: 8 and SEQ ID NO: 9 or an equivalent variant thereof;
a light chain complementarity determining region CDR1′ comprising an amino acid sequence selected from SEQ ID NO: 10, SEQ ID NO: 11 and SEQ ID NO: 12 or an equivalent variant thereof;
a light chain complementarity determining region CDR2′ comprising an amino acid sequence selected from SEQ ID NO: 13, SEQ ID NO: 14 and SEQ ID NO: 15 or an equivalent variant thereof; and
a light chain complementarity determining region CDR3′ comprising an amino acid sequence as shown in SEQ ID NO: 16 or SEQ ID NO: 17 or an equivalent variant thereof;
optionally wherein the complementarity determining regions are in a combination selected from:
(1) a heavy chain complementarity determining region CDR1 comprising an amino acid sequence as shown in SEQ ID NO: 1 or an equivalent variant thereof, a heavy chain complementarity determining region CDR2 comprising an amino acid sequence as shown in SEQ ID NO: 4 or an equivalent variant thereof, a heavy chain complementarity determining region CDR3 comprising an amino acid sequence as shown in SEQ ID NO: 7 or an equivalent variant thereof, a light chain complementarity determining region CDR1′ comprising an amino acid sequence as shown in SEQ ID NO: 10 or an equivalent variant thereof, a light chain complementarity determining region CDR2′ comprising an amino acid sequence as shown in SEQ ID NO: 13 or an equivalent variant thereof, and a light chain complementarity determining region CDR3′ comprising an amino acid sequence as shown in SEQ ID NO: 16 or an equivalent variant thereof;
(2) a heavy chain complementarity determining region CDR1 comprising an amino acid sequence as shown in SEQ ID NO: 2 or an equivalent variant thereof, a heavy chain complementarity determining region CDR2 comprising an amino acid sequence as shown in SEQ ID NO: 5 or an equivalent variant thereof, a heavy chain complementarity determining region CDR3 comprising an amino acid sequence as shown in SEQ ID NO: 8 or an equivalent variant thereof, a light chain complementarity determining region CDR1′ comprising an amino acid sequence as shown in SEQ ID NO: 11 or an equivalent variant thereof, a light chain complementarity determining region CDR2′ comprising an amino acid sequence as shown in SEQ ID NO: 14 or an equivalent variant thereof, and a light chain complementarity determining region CDR3′ comprising an amino acid sequence as shown in SEQ ID NO: 16 or an equivalent variant thereof; and
(3) a heavy chain complementarity determining region CDR1 comprising an amino acid sequence as shown in SEQ ID NO: 3 or an equivalent variant thereof, a heavy chain complementarity determining region CDR2 comprising an amino acid sequence as shown in SEQ ID NO: 6 or an equivalent variant thereof, a heavy chain complementarity determining region CDR3 comprising an amino acid sequence as shown in SEQ ID NO: 9 or an equivalent variant thereof, a light chain complementarity determining region CDR1′ comprising an amino acid sequence as shown in SEQ ID NO: 12 or an equivalent variant thereof, a light chain complementarity determining region CDR2′ comprising an amino acid sequence as shown in SEQ ID NO: 15 or an equivalent variant thereof, and a light chain complementarity determining region CDR3′ comprising an amino acid sequence as shown in SEQ ID NO: 17 or an equivalent variant thereof.

2. (canceled)

3. The bifunctional molecule according to claim 1, wherein the antibody or the antigen-binding fragment thereof further comprises:
a heavy chain framework region FR1 comprising an amino acid sequence selected from SEQ ID NO: 18, SEQ ID NO: 19, SEQ ID NO: 20 and SEQ ID NO: 21 or an equivalent variant thereof;
a heavy chain framework region FR2 comprising an amino acid sequence selected from SEQ ID NO: 22, SEQ ID NO: 23, SEQ ID NO: 24 and SEQ ID NO: 25 or an equivalent variant thereof;
a heavy chain framework region FR3 comprising an amino acid sequence selected from SEQ ID NO: 26, SEQ ID NO: 27, SEQ ID NO: 28 and SEQ ID NO: 29 or an equivalent variant thereof;
a heavy chain framework region FR4 comprising an amino acid sequence as shown in SEQ ID NO: 30 or SEQ ID NO: 31 or an equivalent variant thereof;
a light chain framework region FR1′ comprising an amino acid sequence selected from SEQ ID NO: 32, SEQ ID NO: 33 and SEQ ID NO: 34 or equivalent variant thereof;
a light chain framework region FR2′ comprising an amino acid sequence selected from SEQ ID NO: 35, SEQ ID NO: 36, SEQ ID NO: 37 and SEQ ID NO: 38 or equivalent variant thereof;
a light chain framework region FR3′ comprising an amino acid sequence selected from SEQ ID NO: 39, SEQ ID NO: 40, SEQ ID NO: 41 and SEQ ID NO: 42 or an equivalent variant thereof; and
a light chain framework region FR4′ comprising an amino acid sequence selected from SEQ ID NO: 43, SEQ ID NO: 44 and SEQ ID NO: 45 or equivalent variant thereof;
optionally wherein the framework regions are in a combination selected from:
(1) a heavy chain framework region FR1 comprising an amino acid sequence as shown in SEQ ID NO: 18 or an equivalent variant thereof, a heavy chain framework region FR2 comprising an amino acid sequence as shown in SEQ ID NO: 22 or an equivalent variant thereof, a heavy chain framework region FR3 comprising an amino acid sequence as shown in SEQ ID NO: 26 or an equivalent variant thereof, a heavy chain framework region FR4 comprising an amino acid sequence as shown in SEQ ID NO: 30 or an equivalent variant thereof, a light chain framework region FR1′ comprising an amino acid sequence as shown in SEQ ID NO: 32 or an equivalent variant thereof, a light chain framework region FR2′ comprising an amino acid sequence as shown in SEQ ID NO: 35 or an equivalent variant thereof, a light chain framework region FR3′ comprising an amino acid sequence as shown in SEQ ID NO: 39 or an equivalent variant thereof, and a light chain framework region FR4′ comprising an amino acid sequence as shown in SEQ ID NO: 43 or an equivalent variant thereof;
(2) a heavy chain framework region FR1 comprising an amino acid sequence as shown in SEQ ID NO: 18 or an equivalent variant thereof, a heavy chain framework region FR2 comprising an amino acid sequence as shown in SEQ ID NO: 22 or an equivalent variant thereof, a heavy chain framework region FR3 comprising an amino acid sequence as shown in SEQ ID NO: 26 or an equivalent variant thereof, a heavy chain framework region FR4 comprising an amino acid sequence as shown in SEQ ID NO: 30 or an equivalent variant thereof, a light chain framework region FR1′ comprising an amino acid sequence as shown in SEQ ID NO: 32 or an equivalent variant thereof, a light chain framework region FR2′ comprising an amino acid sequence as shown in SEQ ID NO: 36 or an equivalent variant thereof, a light chain framework region FR3′ comprising an amino acid sequence as shown in SEQ ID NO: 39 or an equivalent variant thereof, and a light chain framework region FR4′ comprising an amino acid sequence as shown in SEQ ID NO: 43 or an equivalent variant thereof;
(3) a heavy chain framework region FR1 comprising an amino acid sequence as shown in SEQ ID NO: 19 or an equivalent variant thereof, a heavy chain framework region FR2 comprising an amino acid sequence as shown in SEQ ID NO: 23 or an equivalent variant thereof, a heavy chain framework region FR3 comprising an amino acid sequence as shown in SEQ ID NO: 27 or an equivalent variant thereof, a heavy chain framework region FR4 comprising an amino acid sequence as shown in SEQ ID NO: 30 or an equivalent variant thereof, a light chain framework region FR1′ comprising an amino acid sequence as shown in SEQ ID NO: 33 or an equivalent variant thereof, a light chain framework region FR2′ comprising an amino acid sequence as shown in SEQ ID NO: 37 or an equivalent variant thereof, a light chain framework region FR3′ comprising an amino acid sequence as shown in SEQ ID NO: 40 or an equivalent variant thereof, and a light chain framework region FR4′ comprising an amino acid sequence as shown in SEQ ID NO: 44 or an equivalent variant thereof;
(4) a heavy chain framework region FR1 comprising an amino acid sequence as shown in SEQ ID NO: 20 or an equivalent variant thereof, a heavy chain framework region FR2 comprising an amino acid sequence as shown in SEQ ID NO: 24 or an equivalent variant thereof, a heavy chain framework region FR3 comprising an amino acid sequence as shown in SEQ ID NO: 28 or an equivalent variant thereof, a heavy chain framework region FR4 comprising an amino acid sequence as shown in SEQ ID NO: 30 or an equivalent variant thereof, a light chain framework region FR1′ comprising an amino acid sequence as shown in SEQ ID NO: 34 or an equivalent variant thereof, a light chain framework region FR2′ comprising an amino acid sequence as shown in SEQ ID NO: 38 or an equivalent variant thereof, a light chain framework region FR3′ comprising an amino acid sequence as shown in SEQ ID NO: 41 or an equivalent variant thereof, and a light chain framework region FR4′ comprising an amino acid sequence as shown in SEQ ID NO: 45 or an equivalent variant thereof; and
(5) a heavy chain framework region FR1 comprising an amino acid sequence as shown in SEQ ID NO: 21 or an equivalent variant thereof, a heavy chain framework region FR2 comprising an amino acid sequence as shown in SEQ ID NO: 25 or an equivalent variant thereof, a heavy chain framework region FR3 comprising an amino acid sequence as shown in SEQ ID NO: 29 or an equivalent variant thereof, a heavy chain framework region FR4 comprising an amino acid sequence as shown in SEQ ID NO: 31 or an equivalent variant thereof, a light chain framework region FR1′ comprising an amino acid sequence as shown in SEQ ID NO: 34 or an equivalent variant thereof, a light chain framework region FR2′ comprising an amino acid sequence as shown in SEQ ID NO: 35 or an equivalent variant thereof, a light chain framework region FR3′ comprising an amino acid sequence as shown in SEQ ID NO: 42 or an equivalent variant thereof, and a light chain framework region FR4′ comprising an amino acid sequence as shown in SEQ ID NO: 44 or an equivalent variant thereof.

4. (canceled)

5. The bifunctional molecule according to claim 1, wherein variable regions of the antibody or the antigen-binding fragment thereof are in a combination selected from:
(1) a heavy chain variable region comprising an amino acid sequence as shown in SEQ ID NO: 46 or an equivalent variant thereof, and a light chain variable region comprising an amino acid sequence as shown in SEQ ID NO: 51 or an equivalent variant thereof;
(2) a heavy chain variable region comprising an amino acid sequence as shown in SEQ ID NO: 46 or an equivalent variant thereof, and a light chain variable region comprising an amino acid sequence as shown in SEQ ID NO: 52 or an equivalent variant thereof;
(3) a heavy chain variable region comprising an amino acid sequence as shown in SEQ ID NO: 47 or an equivalent variant thereof, and a light chain variable region comprising an amino acid sequence as shown in SEQ ID NO: 53 or an equivalent variant thereof;
(4) a heavy chain variable region comprising an amino acid sequence as shown in SEQ ID NO: 48 or an equivalent variant thereof, and a light chain variable region comprising an amino acid sequence as shown in SEQ ID NO: 54 or an equivalent variant thereof;
(5) a heavy chain variable region comprising an amino acid sequence as shown in SEQ ID NO: 49 or an equivalent variant thereof, and a light chain variable region comprising an amino acid sequence as shown in SEQ ID NO: 55 or an equivalent variant thereof; and
(6) a heavy chain variable region comprising an amino acid sequence as shown in SEQ ID NO: 50 or an equivalent variant thereof, and a light chain variable region comprising an amino acid sequence as shown in SEQ ID NO: 56 or an equivalent variant thereof.

6. The bifunctional molecule according to claim 1, wherein for the antibody or the antigen-binding fragment thereof, a heavy chain constant region comprises an amino acid sequence as shown in SEQ ID NO: 57 or an equivalent variant thereof, and a light chain constant region comprises an amino acid sequence as shown in SEQ ID NO: 58 or an equivalent variant thereof.

7. The bifunctional molecule according to claim 1, wherein amino acid sequences of the antibody or the antigen-binding fragment thereof are in a combination selected from:
(1) a heavy chain comprising an amino acid sequence as shown in SEQ ID NO: 59 or an equivalent variant thereof, and a light chain comprising an amino acid sequence as shown in SEQ ID NO: 64 or an equivalent variant thereof;
(2) a heavy chain comprising an amino acid sequence as shown in SEQ ID NO: 59 or an equivalent variant thereof, and a light chain comprising an amino acid sequence as shown in SEQ ID NO: 65 or an equivalent variant thereof;
(3) a heavy chain comprising an amino acid sequence as shown in SEQ ID NO: 60 or an equivalent variant thereof, and a light chain comprising an amino acid sequence as shown in SEQ ID NO: 66 or an equivalent variant thereof;
(4) a heavy chain comprising an amino acid sequence as shown in SEQ ID NO: 61 or an equivalent variant thereof, and a light chain comprising an amino acid sequence as shown in SEQ ID NO: 67 or an equivalent variant thereof;
(5) a heavy chain comprising an amino acid sequence as shown in SEQ ID NO: 62 or an equivalent variant thereof, and a light chain comprising an amino acid sequence as shown in SEQ ID NO: 68 or an equivalent variant thereof; and
(6) a heavy chain comprising an amino acid sequence as shown in SEQ ID NO: 63 or an equivalent variant thereof, and a light chain comprising an amino acid sequence as shown in SEQ ID NO: 69 or an equivalent variant thereof.

8. The bifunctional molecule according to claim 1, wherein:
(a) the antigen binding fragment is selected from scFv, (scFv)₂, Fab, Fab′ or F(ab′)₂ of a PD-L1 antibody;
(b) a C-terminal of the antibody or the antigen-binding fragment thereof is connected with an N-terminal of the human TGFβRII or a functional fragment thereof capable of binding to human TGFβ through a linker, optionally wherein an amino acid sequence of the linker is as shown in SEQ ID NO: 70;
(c) an amino acid sequence of the human TGFβRII is as shown in SEQ ID NO: 7; and/or
(d) the bifunctional molecule has a three-dimensional spatial structure.

9-12. (canceled)

13. A pharmaceutical composition for treating a cancer in a subject, comprising the bifunctional molecule according to claim 1, and a pharmaceutically acceptable carrier or a second therapeutic agent, optionally wherein the cancer is selected from gastric cancer, lung cancer, liver cancer, intrahepatic cholangiocarcinoma, colon cancer, prostate cancer, pancreatic cancer, ovarian cancer, glioma, renal cancer, urothelial cell cancer, breast cancer, cervical cancer, head and neck squamous cell carcinoma, nasopharyngeal cancer, esophageal cancer, bladder cancer, renal cell carcinoma, thyroid cancer, skin cancer and oral squamous cell carcinoma.

14-15. (canceled)

16. A nucleic acid comprising a nucleotide sequence encoding the bifunctional molecule according to claim 1.

17. A vector comprising the nucleotide sequence according to claim 16.

18. A non-human host cell comprising the vector according to claim 17.

19. A monoclonal antibody or an antigen-binding fragment thereof binding to human programmed death-ligand 1 (PD-L1), wherein
a heavy chain complementarity determining region CDR1 comprises an amino acid sequence as shown in SEQ ID NO: 2 or SEQ ID NO: 3 or an equivalent variant thereof;
a heavy chain complementarity determining region CDR2 comprises an amino acid sequence as shown in SEQ ID NO: 5 or SEQ ID NO: 6 or an equivalent variant thereof;
a heavy chain complementarity determining region CDR3 comprises an amino acid sequence as shown in SEQ ID NO: 8 or SEQ ID NO: 9 or an equivalent variant thereof;
a light chain complementarity determining region CDR1′ comprises an amino acid sequence as shown in SEQ ID NO: 11 or SEQ ID NO: 12 or an equivalent variant thereof;
a light chain complementarity determining region CDR2′ comprising an amino acid sequence as shown in SEQ ID NO: 14 or SEQ ID NO: 15 or an equivalent variant thereof; and
a light chain complementarity determining region CDR3′ comprising an amino acid sequence as shown in SEQ ID NO: 16 or SEQ ID NO: 17 or an equivalent variant thereof optionally wherein the heavy chain complementarity determining regions are in a combination selected from:
(1) a CDR1 comprising an amino acid sequence as shown in SEQ ID NO: 2 or an equivalent variant thereof, a CDR2 comprising an amino acid sequence as shown in SEQ ID NO: 5 or an equivalent variant thereof, and a CDR3 comprising an amino acid sequence as shown in SEQ ID NO: 8 or an equivalent variant thereof; and
(2) a CDR1 comprising an amino acid sequence as shown in SEQ ID NO: 3 or an equivalent variant thereof, a CDR2 comprising an amino acid sequence as shown in SEQ ID NO: 6 or an equivalent variant thereof, and a CDR3 comprising an amino acid sequence as shown in SEQ ID NO: 9 or an equivalent variant thereof; and
the light chain complementarity determining regions are in a combination selected from:
(1) a CDR1′ comprising an amino acid sequence as shown in SEQ ID NO: 11 or an equivalent variant thereof, a CDR2′ comprising an amino acid sequence as shown in SEQ ID NO: 14 or an equivalent variant thereof, and a CDR3′ comprising an amino acid sequence as shown in SEQ ID NO: 16 or an equivalent variant thereof, and
(2) a CDR1′ comprising an amino acid sequence as shown in SEQ ID NO: 12 or an equivalent variant thereof, a CDR2′ comprising an amino acid sequence as shown in SEQ ID NO: 15 or an equivalent variant thereof, and a CDR3′ comprising an amino acid sequence as shown in SEQ ID NO: 17 or an equivalent variant thereof.

20. (canceled)

21. The monoclonal antibody or an antigen-binding fragment thereof according to claim 19, further comprising:
a heavy chain framework region FR1 comprising an amino acid sequence as shown in SEQ ID NO: 18 or SEQ ID NO: 19 or an equivalent variant thereof;
a heavy chain framework region FR2 comprising an amino acid sequence as shown in SEQ ID NO: 22 or SEQ ID NO: 23 or an equivalent variant thereof;
a heavy chain framework region FR3 comprising an amino acid sequence as shown in SEQ ID NO: 26 or SEQ ID NO: 27 or an equivalent variant thereof;
a heavy chain framework region FR4 comprising an amino acid sequence as shown in SEQ ID NO: 30 or an equivalent variant thereof;
a light chain framework region FR1′ comprising an amino acid sequence as shown in SEQ ID NO: 32 or SEQ ID NO: 33 or an equivalent variant thereof;
a light chain framework region FR2′ comprising an amino acid sequence as shown in SEQ ID NO: 36 or SEQ ID NO: 37 or an equivalent variant thereof;
a light chain framework region FR3′ comprising an amino acid sequence as shown in SEQ ID NO: 39 or SEQ ID NO: 40 or an equivalent variant thereof; and
a light chain framework region FR4′ comprising an amino acid sequence as shown in SEQ ID NO: 43 or SEQ ID NO: 44 or an equivalent variant thereof;
optionally wherein the framework regions are in a combination selected from:
(1) a heavy chain framework region FR1 comprising an amino acid sequence as shown in SEQ ID NO: 18 or an equivalent variant thereof, a heavy chain framework region FR2 comprising an amino acid sequence as shown in SEQ ID NO: 22 or an equivalent variant thereof, a heavy chain framework region FR3 comprising an amino acid sequence as shown in SEQ ID NO: 26 or an equivalent variant thereof, a heavy chain framework region FR4 comprising an amino acid sequence as shown in SEQ ID NO: 30 or an equivalent variant thereof, a light chain framework region FR1′ comprising an amino acid sequence as shown in SEQ ID NO: 32 or an equivalent variant thereof, a light chain framework region FR2′ comprising an amino acid sequence as shown in SEQ ID NO: 36 or an equivalent variant thereof, a light chain framework region FR3′ comprising an amino acid sequence as shown in SEQ ID NO: 39 or an equivalent variant thereof, and a light chain framework region FR4′ comprising an amino acid sequence as shown in SEQ ID NO: 43 or an equivalent variant thereof; and
(2) a heavy chain framework region FR1 comprising an amino acid sequence as shown in SEQ ID NO: 19 or an equivalent variant thereof, a heavy chain framework region FR2 comprising an amino acid sequence as shown in SEQ ID NO: 23 or an equivalent variant thereof, a heavy chain framework region FR3 comprising an amino acid sequence as shown in SEQ ID NO: 27 or an equivalent variant thereof, a heavy chain framework region FR4 comprising an amino acid sequence as shown in SEQ ID NO: 30 or an equivalent variant thereof, a light chain framework region FR1′ comprising an amino acid sequence as shown in SEQ ID NO: 33 or an equivalent variant thereof, a light chain framework region FR2′ comprising an amino acid sequence as shown in SEQ ID NO: 37 or an equivalent variant thereof, a light chain framework region FR3′ comprising an amino acid sequence as shown in SEQ ID NO: 40 or an equivalent variant thereof, and a light chain framework region FR4′ comprising an amino acid sequence as shown in SEQ ID NO: 44 or an equivalent variant thereof.

22. (canceled)

23. The monoclonal antibody or an antigen-binding fragment thereof according to claim 19, wherein
a heavy chain variable region comprises an amino acid sequence as shown in SEQ ID NO: 47 or SEQ ID NO: 48 or an equivalent variant thereof; and
a light chain variable region comprises an amino acid sequence as shown in SEQ ID NO: 53 or SEQ ID NO: 54 or an equivalent variant thereof;
optionally wherein the variable regions are in a combination selected from:
(1) a heavy chain variable region comprising an amino acid sequence as shown in SEQ ID NO: 47 or an equivalent variant thereof, and a light chain variable region comprising an amino acid sequence as shown in SEQ ID NO: 53 or an equivalent variant thereof; and
(2) a heavy chain variable region comprising an amino acid sequence as shown in SEQ ID NO: 48 or an equivalent variant thereof, and a light chain variable region comprising an amino acid sequence as shown in SEQ ID NO: 54 or an equivalent variant thereof.

24. (canceled)

25. The monoclonal antibody or an antigen-binding fragment thereof according to claim 19, wherein a heavy chain constant region comprises an amino acid sequence as shown in SEQ ID NO: 57 or an equivalent variant thereof, and a light chain constant region comprises an amino acid sequence as shown in SEQ ID NO: 58 or an equivalent variant thereof.

26. The monoclonal antibody or an antigen-binding fragment thereof according to claim 19, wherein a heavy chain comprises an amino acid sequence as shown in SEQ ID NO: 60 or SEQ ID NO: 61 or an equivalent variant thereof, and a light chain comprises an amino acid sequence as shown in SEQ ID NO: 66 or SEQ ID NO: 67 or an equivalent variant thereof;
optionally wherein the heavy chain and the light chain are in a combination selected from:
(1) a heavy chain comprising an amino acid sequence as shown in SEQ ID NO: 60 or an equivalent variant thereof, and a light chain comprising an amino acid sequence as shown in SEQ ID NO: 66 or an equivalent variant thereof; and
(2) a heavy chain comprising an amino acid sequence as shown in SEQ ID NO: 61 or an equivalent variant thereof, and a light chain comprising an amino acid sequence as shown in SEQ ID NO: 67 or an equivalent variant thereof.

27. (canceled)

28. The monoclonal antibody or an antigen-binding fragment thereof according to claim 19, wherein the antigen-binding fragment is selected from scFv, (scFv)₂, Fab, Fab′ or F(ab′)₂ of a PD-L1 antibody.

29. A pharmaceutical composition for treating a cancer in a subject, comprising the monoclonal antibody or an antigen-binding fragment thereof according to claim 19, and a pharmaceutically acceptable carrier or a second therapeutic agent, optionally wherein the cancer is selected from gastric cancer, lung cancer, liver cancer, intrahepatic cholangiocarcinoma, colon cancer, pancreatic cancer, ovarian cancer, glioma, renal cancer, urothelial cell cancer, breast cancer, cervical cancer, head and neck squamous cell carcinoma, nasopharyngeal cancer, esophageal cancer, bladder cancer, renal cell carcinoma, skin cancer and oral squamous cell carcinoma.

30-31. (canceled)

32. A nucleotide sequence encoding the monoclonal antibody or an antigen-binding fragment thereof according to claim 19.

33. A vector comprising the nucleotide sequence according to claim 32.

34. A non-human host cell comprising the vector according to claim 33.
